(19) United States
(12) Patent Application Publication
(10) Pub. No.: US 2012/0178793 A1
(43) Pub. Date: Jul. 12, 2012

(54) NUCLEOTIDE-COCHLEATE COMPOSITIONS AND METHODS OF USE

(75) Inventors: Raphael J. Mannino, Annandale, NJ (US); Susan Gould-Fogerite, Annandale, NJ (US)

(73) Assignee: BioDelivery Sciences International, Inc., Morrisville, NC (US)

(21) Appl. No.: 13/424,282

(22) Filed: Mar. 19, 2012

Related U.S. Application Data
(63) Continuation of application No. 11/578,187, filed on Jan. 24, 2008, now abandoned.
Application No. 11/578,187 is a continuation of application No. 10/822,235, filed on Apr. 9, 2004, now abandoned.
(60) Provisional application No. 60/623,097, filed on Oct. 27, 2004.
(60) Provisional application No. 60/656,115, filed on Feb. 23, 2005.

(51) Int. Cl. A61K 31/713; A61P 31/04; A61P 31/18; A61P 31/10; A61P 35/00; A61P 31/12

(52) U.S. Cl. 514/44 A

(57) ABSTRACT

The present invention is directed to cochleate composition that include a nucleotide. The nucleotide may generally be bound via a linker to a component of the cochleate, or to a lipophilic tail. Additionally or alternatively, the nucleotide may he associated with a transfection agent. The present invention also includes methods for making and using, the compositions provided herein.

### RELATED APPLICATIONS

This application claims the benefit of and priority to U.S. Utility application Ser. No. 10/822,235, filed Apr. 9, 2004; International Application No. PCT US2004/011020, filed Apr. 9, 2004; U.S. Provisional Application No. 60/623,097, filed Oct. 27, 2004; and U.S. Provisional Application Ser. No. 60/656,115, filed Feb. 23, 2005. The entire contents of each of the aforementioned applications are hereby expressly incorporated herein by reference in their entireties.

### BACKGROUND OF THE INVENTION

Cochleate structures were first prepared by D. Papahadjopoulos as an intermediate in the preparation of large unilamellar vesicles. U.S. Pat. No. 4,078,052. Cochleate compositions incorporating a variety of cargo moieties, including methods of making and using such cochleates have also been disclosed, e.g., in U.S. Pat. Nos. 5,840,707, 5,994,318, and 6,153,217, and International Application No. WO 04/091578. Specifically, U.S. Pat. No. 5,840,707 discloses protein-cochleates and polynucleotide-cochleates. The entire contents of these patents are incorporated by this reference.

Additionally, lipid tails bound to peptides have been disclosed, e.g., in Bourgault, I., F. Chirat, A. Tartar, J.-P. Levy, J. G. Guillet, and A. Venet, J. Immunol. 152:2530-2537 (1994), and have been shown to induce CD8⁺ MHC class I-restricted CTL in vitro and in vivo.

### SUMMARY OF THE INVENTION

The present invention is generally directed to nucleotide-cochleate compositions and methods of manufacture and administration. The composition may generally include a cochleate and a nucleotide, wherein the nucleotide is bound to a cochleate component via a linker. Alternatively or additionally, the composition may generally include a cochleate and a nucleotide associated with the cochleate, wherein the nucleotide is bound to a lipophilic tail via a linker.

In some embodiments, the linker is lipophilic or hydrophobic. In some embodiments, the linker stabilizes the nucleotide. In other embodiments, the linker facilitates association of the nucleotide with the cochleate component. In still other embodiments, the linker is digestible, reducible, or otherwise reversible. The linker can be, but is not limited to SMPB and/or SPDP.

In some embodiments, the cochleate includes a negatively charged lipid component and a multivalent cation component. In other embodiments, the cochleate includes soy phosphatidylserine.

In some embodiments, the nucleotide can be, but is not limited to an siRNA, a morpholino oligonucleotide, e.g., an antisense morpholino oligonucleotide, a short double-stranded DNA, a ribozyme, an aptamer, and/or a transcription factor decoy. In some embodiments, the nucleotide includes at least one mismatch. In other embodiments, the nucleotide includes at least one substitution. In certain embodiments, the nucleotide is about 18-25 nucleotides long. In other embodiments, the nucleotide is about 21-23 nucleotides long.

In some embodiments, the nucleotide mediates RNA interference against a target mRNA. In other embodiments, the nucleotide mediates inhibition of translation of a target mRNA. The target mRNA expresses a protein which can be, but is not limited to a cancer protein, a virus protein, an HIV protein, a fungus protein, a bacterial protein, an abnormal cellular protein, and/or a normal cellular protein. In some embodiments, the composition further includes a second nucleotide directed against a second target mRNA.

In some embodiments, the nucleotide is complexed with a transfection agent prior to contacting a liposome. In certain embodiments, the transfection agent is a polycationic transfection agent. The transfection agent can be, but is not limited to, polyethylenimine (PEI), protamine, and/or derivatives thereof.

In another aspect, the present invention provides a nucleotide-cochleate composition that includes a cochleate and a nucleotide associated with the cochleate, wherein the nucleotide is complexed with a transfection agent. In certain embodiments, the transfection agent is a polycationic transfection agent. The transfection agent can be, but is not limited to, polyethylenimine (PEI), protamine, and/or derivatives thereof.

In some embodiments, the nucleotide complexed with the transfection agent is associated with the cochleate or a lipid tail via a linker. In some embodiments, the nucleotide can be, but is not limited to an siRNA, a morpholino oligonucleotide, e.g., an antisense morpholino oligonucleotide, a short double-stranded DNA, a ribozyme, an aptamer, and/or a transcription factor decoy. In some embodiments, the nucleotide includes at least one mismatch. In other embodiments, the nucleotide includes at least one substitution. In certain embodiments, the nucleotide is about 18-25 nucleotides long. In other embodiments, the nucleotide is about 21-23 nucleotides long.

In some embodiments, the nucleotide mediates RNA interference against a target mRNA. In other embodiments, the nucleotide mediates inhibition of translation of a target mRNA. The target mRNA expresses a protein which can be, but is not limited to a cancer protein, a virus protein, an HIV protein, a fungus protein, a bacterial protein, an abnormal cellular protein, and/or a normal cellular protein. In some embodiments, the composition further includes a second nucleotide directed against a second target mRNA.

In some aspects, the present invention is directed to a method of forming nucleotide-cochleate compositions. The method generally includes precipitating a liposome and a nucleotide to form a nucleotide-cochleate, wherein the nucleotide is any of the nucleotides described herein.

In other aspects, the present invention is directed to a method of administering a nucleotide to a host. The method generally includes administering a biologically effective amount of a nucleotide-cochleate composition to a host comprising a cochleate and a nucleotide associated with the cochleate, wherein the nucleotide is any of the nucleotides described herein.

In other aspects, the present invention is directed to a method of treating a subject having a disease or disorder associated with expression of a target mRNA. The method generally includes administering to a subject a therapeutically effective amount of a nucleotide-cochleate composition, comprising a cochleate and a nucleotide directed against a target mRNA associated with a disease or disorder, wherein the nucleotide is any of the nucleotides described herein, such that the disease or disorder is treated.

### BRIEF DESCRIPTION OF THE DRAWINGS

FIG. 1 is a graph indicating the partial knockdown effect of anti-erb B2 siRNA-cochleates formulated with PEI, and washed to remove free siRNA, on SKOV3 cells.

FIG. 2 is a graph indicating indicated increased RNAi effect in encochleated siRNA versus unencochleated siRNA in SKOV3 cells.

FIGS. 3-7 are images showing GFP expression in two tumour models in mice.

FIGS. 8-9 are graphs of relative fluorescence intensity and cell viability data of different plasmid DNA formulations in SKOV3 cells.

### DETAILED DESCRIPTION OF THE INVENTION

The present invention is based, at least in part, on the discovery that employing a nucleotide associated with a cochleate component or a lipophilic tail may increase association with a cochleate and facilitate bioavailability of the nucleotide.

Accordingly, in some aspects, the present invention provides nucleotide-cochleate compositions which include a cochleate component and a nucleotide. In certain embodiments of the present invention, the nucleotide is bound to the cochleate component via a linker. In other embodiments of the present invention, the nucleotide is bound to a lipophilic tail via a linker.

So that the invention may be more readily understood, certain terms are first defined.

The term “lipophilic tail,” as used herein, refers to a moiety that demonstrates one or more of the following characteristics: tends to be water insoluble, tends to be soluble in non-polar solvent, tends to favor octanol in octanol/water partition measurements, or tends to be compatible with lipid bilayers and may be bilayer forming. Accordingly, lipophilic tails include moieties which may be completely hydrophobic, predominantly hydrophobic, or may be partially hydrophilic.

As used herein, the terms “cochleate,” “lipid precipitate” and “precipitate” are used interchangeably to refer to a lipid precipitate component that generally includes alternating cationic and lipid bilayer sheets with little or no internal aqueous space, typically stacked and/or rolled up, wherein the cationic sheet is comprised of one or more multivalent cations. Additionally, the term “encochleated” means associated with the cochleate structure, e.g., by incorporation into the cationic sheet, and/or inclusion in the lipid bilayer.

As used herein, the term “multivalent cation” refers to a divalent cation or higher valency cation, or any compound that has at least two positive charges, including mineral cations such as calcium, barium, zinc, iron and magnesium and other elements, such as drugs and other compounds, capable of forming ions or other structures having multiple positive charges capable of chelating and bridging negatively charged lipids. Additionally or alternatively, the multivalent cation can include other multivalent cationic compounds, e.g., cationic or protonized cargo moieties.

The term “nucleoside” refers to a molecule having a purine or pyrimidine base covalently linked to a ribose or deoxyribose sugar. Exemplary nucleosides include adenosine, guanosine, cytidine, uridine and thymidine. The term “nucleotide” refers to a nucleoside having one or more phosphate groups joined in ester linkages to the sugar moiety. Exemplary nucleotides include nucleoside monophosphates, diphosphates and triphosphates. The terms “polynucleotide” and “nucleic acid molecule” are used interchangeably herein and refer to a polymer of nucleotides joined together by a phosphodiester linkage between 5′ and 3′ carbon atoms.

The term “oligonucleotide” refers to a short sequence of nucleotides and/or nucleotide analogs. The term “RNA analog” refers to an polynucleotide (e.g., a chemically synthesized polynucleotide) having at least one altered or modified nucleotide as compared to a corresponding unaltered or unmodified RNA, but retaining the same or similar nature or function as the corresponding unaltered or unmodified RNA. As discussed herein, the oligonucleotides may be linked with linkages which result in a lower rate of hydrolysis of the RNA analog as compared to an RNA molecule with phosphodiester linkages. For example, the nucleotides of the analog may comprise methylenediol, ethylene diol, oxymethylthio, oxyethylthio, oxycarbonyloxy, phosphorodiamidate, phosphoroamidate, and/or phosphorothioate linkages. Preferred RNA analogues include sugar- and/or backbone-modified ribonucleotides and/or deoxyribonucleotides. Such alterations or modifications can further include addition of non-nucleotide material, such as to the end(s) of the RNA or internally (at one or more nucleotides of the RNA). An RNA analog need only be sufficiently similar to natural RNA that it has the ability to mediate (mediates) RNA interference.

As used herein, an “identical” oligonucleotide has the same sequence as the reference nucleotide subsequence to which the oligonucleotide is being compared. An “exactly complementary” oligonucleotide refers to an oligonucleotide whose complement has the same sequence as the reference nucleotide subsequence to which the oligonucleotide is being compared. A “substantially complementary” and a “substantially identical” oligonucleotide have the ability to specifically hybridize to a reference gene, DNA, cDNA, or mRNA, and its exact complement.

An “antisense” oligonucleotide is an oligonucleotide that is substantially complementary to a target nucleotide sequence and has the ability to specifically hybridize to the target nucleotide sequence.

The term “RNA” or “RNA molecule” or “ribonucleic acid molecule” refers to a polymer of ribonucleotides. The term “DNA” or “DNA molecule” or deoxyribonucleic acid molecule” refers to a polymer of deoxyribonucleotides. DNA and RNA can be synthesized naturally (e.g., by DNA replication or transcription of DNA, respectively). RNA can be post-transcriptionally modified. DNA and RNA can also be chemically synthesized. DNA and RNA can be single-stranded (i.e., ssRNA and ssDNA, respectively) or multi-stranded (e.g., double stranded, i.e., dsRNA and dsDNA, respectively). “mRNA” or “messenger RNA” is single-stranded RNA that specifies the amino acid sequence of one or more polypeptide chains. This information is translated during protein synthesis when ribosomes bind to the mRNA.

As used herein, the term “RNA interference” (“RNAi”) refers to a selective intracellular degradation of RNA to mediate, reduce or silence the expression of a target gene.

As used herein, the term “small interfering RNA” (“siRNA”) (also referred to in the art as “short interfering RNAs”) refers to a double stranded RNA (or RNA analog) comprising between about 10-50 nucleotides (or nucleotide analogs) which is capable of directing or mediating RNA interference.

As used herein, the term “short double stranded DNA” refers to a double stranded DNA (or DNA analog) comprising less than about 50 nucleotides (or nucleotide analogs).

“Morpholino oligonucleotides” and “morpholinos” are used interchangeably, and refer to oligonucleotides having a morpholino backbone. Morpholinos function by an RNase H-independent mechanism and are soluble in aqueous solutions, with most being freely soluble at mM concentrations (typically 10 mg/ml to over 100 mg/ml). Morpholinos have high affinity for RNA and efficiently invade even quite stable secondary structures in mRNAs, which results in effective and predictable targeting essentially anywhere from the 5′-cap to about +25 of the protein coding region of mRNAs. Morpholinos are free of significant non-antisense effects while the alternative phosphorothioates are plagued by a host of well-documented non-antisense effects. Morpholinos include a morpholine backbone, which is not recognized by nucleases and therefore is stable in the cell compared to phosphorothioates, which typically are degraded in biological systems in a matter of hours. Consequently, considerably fewer morpholinos are required (approximately 100× less) to achieve similar antisense effects. Morpholinos also are superior to phosphorothioates because targeting is more predictable, the activity in cells is more reliable, and the sequence specificity is superior. Summerton, Biochimica et Biophysica Acta 1489: 141-158 (1999). Morpholinos can be designed and prepared according to known methods. E.g., Summerton and Weller, Antisense and Nucleic Acid Drug Development 7187-195 (1997).

As used herein, the terms “ribozyme” and “RNA enzyme” are used interchangeably to refer to RNA molecules that catalyze chemical reactions. In addition to catalyzing cleavage of themselves and/or other RNAs, ribozymes may also catalyze the aminotransferase activity of the ribosome. Ribozymes, although quite rare in cells, often have essential functions, e.g., a role in the ribosomal translation of RNA into proteins. Known ribozymes include, but are not limited to RNase P, Group I and Group II introns, leadzyme, hairpin ribozyme, hammerhead ribozyme, hepatitis delta virus ribozyme, and tetrahymena ribozyme. Additionally, ribosymes may be made synthetically, e.g., while maintaining good enzymatic activity. Synthetic ribozymes may have structures similar to naturally occurring ribozymes or may have novel structures.

The term “aptamer,” as used herein, refers generally to single-stranded DNA and RNA molecules that bind target molecules with high affinity and specificity. Aptamers may be selected in vitro from populations of random sequences that recognize specific ligands by forming binding pockets. Aptamers can be chemically synthesized and, although single-stranded, normally have complex three-dimensional shapes. Generally, an aptamer domain on an RNA enzyme, or ribozyme, modulates the activity of the ribozyme. In a manner similar to antibodies, when the shape of the aptamer corresponds to the shape of a target protein, a strongly bound complex can be formed. These aptamers may have potential in targeted delivery of drugs, either through direct conjugation of the drug to an aptamer, or through drug encapsulation in a vesicle, e.g., a liposome, which is coated in an aptamer. Accordingly, in one embodiment, the nucleotides employed in the compositions of the present invention are aptamers. The aptamers may be contained at least partially within the cochleate structure. Alternatively, the aptamers may be coated on the cochleate. The aptamer-cochleate may include additional cargo moieties for targeted delivery.

As used herein, the term “transcription factor decoys” refers to nucleotides, generally oligodeoxynucleotides (ODNs), which are used to inhibit specific transcription factors, e.g., in cell culture. Transcription factor proteins bind specific sequences found in the promoter regions of target genes whose expression they then regulate. These binding sequences are generally 6-10 base pairs in length and are occasionally found in multiple copies within the promoter regions of target genes. A cell can be flooded with transcription factor decoys, which compete for binding of the transcription factor with sequences in target genes. The decoys have the potential to alter the binding and function of the transcription factor, thus regulating the expression of the target gene. At higher concentrations, transcription factor decoys may completely block transcription factor function. Exemplary transcription factor decoys are described, e.g., in Morishita, R., et al., Circ Res, 82, 1023-1028 (1998) and Mann, M. J. and Dzau, V. J., J. Clin. Invest., 106, 1071-1075 (2000).

A nucleotide “that mediates RNAi against a target mRNA” refers to a nucleotide including a sequence sufficiently complementary to a target RNA (e.g. mRNA or RNA that can be spliced to produce one or more mRNAs) to trigger the destruction of the target mRNA by the RNAi machinery or process or to interfere with translation of the mRNA into a protein.

The term “nucleotide analog” or “altered nucleotide” or “modified nucleotide” refers to a non-standard nucleotide, including non-naturally occurring ribonucleotides or deoxyribonucleotides. Preferred nucleotide analogs are modified at any position so as to alter certain chemical properties of the nucleotide yet retain the ability of the nucleotide analog to perform its intended function. Examples of positions of the nucleotide which may be derivatized include the 5 position, e.g., 5-(2-amino)propyl uridine, 5-bromo uridine, 5-propyne uridine, 5-propenyl uridine, etc.; the 6 position, e.g., 6-(2amino)propyl uridine; the 8-position for adenosine and/or guanosines, e.g., 8-bromo guanosine, 8-chloro guanosine, 8-fluoroguanosine, etc. Nucleotide analogs also include deaza nucleotides, e.g., 7-deaza-adenosine; and N-modified (e.g., alkylated, e.g., N6methyl adenosine, or as otherwise known in the art) nucleotides; and other heterocyclically modified nucleotide analogs such as those described in Herdewijn, Antisense Nucleic Acid Drug Dev., 2000 Aug. 10(4):297-310.

Nucleotide analogs may also comprise modifications to the sugar portion of the nucleotides. For example the 2′OH-group may be replaced by a group selected from H, OR, R, F, Cl, Br, I, SH, SR, NH₂, NHR, NR₂, COOR, or OR, wherein R is substituted or unsubstituted C1-C6 alkyl, alkenyl, alkynyl, aryl, etc. Other possible modifications include those described in U.S. Pat. Nos. 5,858,988, and 6,291,438.

The phosphate group of the nucleotide may also be modified, e.g., by substituting one or more of the oxygens of the phosphate group with sulfur (e.g., phosphorothioates), or by making other substitutions which allow the nucleotide to perform its intended function such as described in, for example, Eckstein, Antisense Nucleic Acid Drug Dev. 2000 Apr. 10(2):117-21, Rusckowski et al. Antisense Nucleic Acid Drug Dev. 2000 Oct. 10(5):333-45, Stein, Antisense Nucleic Acid Drug Dev. 2001 Oct. 11 (5): 317-25, Vorobjev et al. Antisense Nucleic Acid Drug Dev. 2001 April 11.2):77-85, and U.S. Pat. No. 5,684,143. Certain of the above-referenced modifications (e.g., phosphate group modifications) preferably decrease the rate of hydrolysis of, for example, polynucleotides comprising said analogs in vivo or in vivo.

A gene or mRNA “involved” in or “associated with” a disorder includes a gene or mRNA, the normal or aberrant expression or function of which effects or causes a disease or disorder or at least one symptom of said disease or disorder.

The phrase “examining the function of a target mRNA” refers to examining or studying the expression, activity, function or phenotype arising therefrom, in the host cell, tissue or organism.

The cochleates of the present invention include nucleotides which are bound to or associated with a lipophilic tail (e.g., any of the lipids described herein, polyethylenimine, or Vitamin E) or a cochleate component via a linker. Binding or otherwise associating the nucleotide to a cochleate component or to a lipophilic tail may be advantageous, e.g., for facilitating incorporation of the nucleotide into the cochleate, for facilitating the transfer of the nucleotide across membranes subsequent to administration, or both. In some embodiments, the cross-linker linking the nucleotide and the lipophilic tail is also lipophilic or hydrophobic. Such cross-linkers may further facilitate incorporation of the nucleotide into the cochleate. In some embodiments, the cross-linker is stable in vivo and/or in vitro. In some embodiments the cross-linker is unstable in vivo and/or in vitro. In some embodiments, the lipophilic tail and/or cross-linker stabilize the nucleotide. In some embodiments, the lipophilic tail and/or cross-linker stabilizes a nucleotide duplex or other complex (e.g., with a transfection agent). In some embodiments, the lipophilic tail and/or cross-linker increases or decreases the ability of the nucleotide, e.g., an siRNA, to modulate RNAi, e.g., by stabilizing or interfering with the RNAi mechanism. In some embodiments, cochleates including nucleotides with lipophilic tails have significantly no toxicity or undetectable toxicity in vivo and/or in vitro. In one embodiment, the nucleotide is bound to the lipid cochleate component or the lipophilic tail with a digestible, reducible, or otherwise reversible linker. The nucleotide can be bound in a reversible manner (e.g., with a reducible or digestible linker) or a linker susceptible to target conditions (e.g., pH, temperature, ultrasonic energy and the like). This is particularly useful as the linker can be chosen such that it is readily digestible, e.g., by an enzyme, in the body generally or even in a target structure. Thus, e.g., a linker can be chosen such that it is degraded by an enzyme in the plasma, interstitial fluids, in a cell (e.g. a macrophage) or in an endosome, such that the nucleotide becomes detached and available in unbound form in these structures. In another embodiment, the reversible linker can be an electrostatic or other bond that is broken by a change in pH, e.g., in an organ or other structure in which the cochleate experiences a pH gradient. In another embodiment, the linker is reversed by a change in temperature, e.g., by exposure to body temperature.

In one embodiment, the nucleotide is bound by an electrostatic, hydrophobic, covalent, or ionic interaction with a lipid component such as a lipophilic tail. In a preferred embodiment, the nucleotide is bound to a component of the bilayer of the cochleate, e.g., a phospholipid or other lipid. Covalently binding the nucleotide to the lipid by cross-linking can be accomplished by known methods. For example, covalently binding a nucleotide to the lipid by cross-linking can be accomplished by methods using N-succinimidyl-4-(p-maleimidophenyl)butyrate (SMPB), a stable cross-linker, or N-hydroxysuccinimidyl 3-(2-pyridyldithio)propionate (SPDP), a reducible cross-linker. See, e.g., Martin, F. J., and Papahadjopoulos, D. J. Bio. Chem. 257:286-288 (1982) and Barbet, J. et al. J. Supra. Struct. Cell Biochem. 16:243-258 (1981).

SMPB features an extended chain length to limit steric hindrance. It is also an extended chain analog of MBS, it generally forms conjugates which can be shown to be more stable in serum than SPDP conjugates, and SMPB's reactive groups include NHS ester and maleimide groups. Additionally, SMPB is generally reactive towards amino and sulfhydryl groups. SMPB can be found, e.g., in Iwai, K., et al., Anal. Biochem. 171, 277-282 (1988).

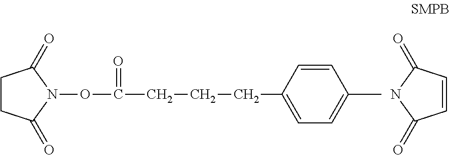

SPDP is a classic heterobifunctional, cleavable cross-linker. It is widely used in immunochemistry and conjugates used in drug carrier systems, antibody production and enzyme immunoassays have been successfully prepared with SPDP. SPDP can be used as a protein thiolation reagent, resulting in available —SH groups, and SPDP's reactive groups include NHS ester and pyridyldithio groups. Additionally, SPDP is generally reactive towards amino and sulfhydryl groups. SPDP can be found, e.g., in Carlsson, J., et al., Biochem. J. 173, 723-737 (1978).

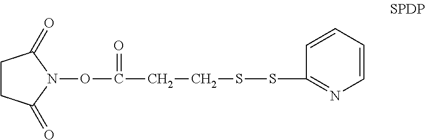

In one embodiment, the covalent bond is reversible so that the nucleotide can be detached from the lipid component or lipophilic tail under suitable conditions. For example, a nucleotide can be attached to a phospholipid via a linker that can be cleaved by an enzyme endogenous to a target tissue, organ, or structure (e.g., a plasma protein, interstitial protein, an endosome or the intracellular milieu), such that the nucleotide is delivered to the target tissue, organ or other structure. In alternative embodiments the nucleotide can be attached by any other means, for example, by electrostatic interactions and/or hydrophobic interactions.

The nucleotide can be associated with the lipid component or lipophilic tail in any of the methods described herein. For example, in one embodiment, the nucleotide is associated with the lipid component, such that the nucleotide dissociates with the lipid component upon contact with a target environment. The nucleotide can be bound to a component of the cochleate with any of the linkers described herein, e.g., a linker that is reducible, or otherwise reversible or digestible by an enzyme, protein, or molecule endogenous to the target environment. The enzyme can be an extracellular, intracellular or endosomal enzyme endogenous to the subject. In another embodiment, the nucleotide component is electrostatically associated with the lipid component and dissociates with the cochleate upon contact with a pH gradient in a cell or organ of the subject.

In other aspects, the present invention provides nucleotide-cochleate compositions which include a cochleate component and a nucleotide, wherein the nucleotide is complexed to a transfection-agent.

Enhanced binding of the nucleotide and the liposome and/or cochleates may be achieved by first forming a complex between the nucleotide and a transfection agent. Transfection agents may be cationic, or polycationic, e.g., protamine, polyethylenimine (PEI), polyvinylamine, spermine, spermidine, histamine, cationic lipid, or other moieties which enhance binding to the liposome prior to precipitation. Alternatively the polycation is mixed with and binds to the liposome first and then the nucleotide is added. The high transfection potential of DNA complexed with the cationic polymer polyethylenimine (PEI) has been described. Boussif et al. Proc Natl Acad Sci USA 92: 7297-7301 (1995). However, increased transfection rates have been coupled with increased toxicity. Bogden et al., AACS PharmSci 4(2) (2002). PEI can be obtained, e.g., from BASF, such as that sold under the trade name Lupasol G35. Additionally, the use of protamine sulfate to condense plasmid DNA, thus enhancing lipid-mediated gene transfer, has been described, e.g., in Sorgi F. L., et al., Gene Ther. 4(9):961-8 (1997). Cationic polymers may be employed having a variety of molecular weights, and may be branched or unbranched.

The addition of a transfection agent to a nucleotide cochleate may be advantageous, e.g., in delivery of the nucleotide. For example, it has been discovered that encochleated siRNA-PEI complexes improve transfection into cells without the associated toxicity observed in the literature. In some embodiments the cation is a cationic polymer, e.g., PEI or PEI derivatives. In other embodiments, the cation is protamine. Such complexes can be associated with the liposomes by any of the methods discussed herein.

The ratios of lipid to nucleotide, and PEI or protamine to nucleotide, etc. may vary. In a preferred embodiment, N to P ratios (N, nitrogen in PEI or protamine to P, phosphate in the nucleotide) may vary from between about 0.5 to about 20. In certain embodiments, the N to P ration is between about 4 and about 8.

In certain aspects, the present invention features encochleated nucleotide compositions. The nucleotide-cochleate compositions generally include a cochleate, and a nucleotide as described herein associated with the cochleate, e.g., a nucleotide that is bound to a lipophilic tail via a linker. The present invention also features methods (e.g., research and/or therapeutic methods) for using nucleotide-cochleates.

In some embodiments, the nucleotide is an siRNA. In other embodiments, the nucleotide is a morpholino oligonucleotide. Morpholino oligonucleotides suitable for use in the present invention include antisense morpholino oligonucleotides. In still other embodiments, the nucleotide is a short double-stranded DNA. In still other embodiments, the nucleotide is a ribozyme. In yet other embodiments, the nucleotide is an aptamer. In still other embodiments, the nucleotide is a transcription factor decoy. In certain embodiments of the present invention, the nucleotide is not DNA.

The nucleotides of the present invention can be between about 7 and 100 nucleotides long, between 10 and 50, between 20 and 35, and between 15 and 30 nucleotides long. The nucleotides of the present invention can be 15, 16, 17, 18, 19, 20, 21, 22, 23, 24, 25, 26, 27, 28, 29 or 30 nucleotides in length. For example, in some embodiments, a morpholino oligonucleotide is between about 18 and about 25 nucleotides long. In other embodiments, an siRNA has a length of from about 21-23 nucleotides.

The nucleotides of the invention, e.g., siRNAs or morpholinos, can mediate RNA interference against a target gene. That is, in some embodiments, the nucleotide has a sequence sufficiently complementary to a target RNA (e.g. mRNA or RNA that can be spliced to produce one or more mRNAs) associated with a target gene to trigger the destruction of the target mRNA by the RNAi machinery or process. The nucleotide can be designed such that every residue is complementary to a residue in the target molecule. Alternatively, one or more substitutions can be made within the molecule to increase stability and/or enhance processing activity of said molecule. Substitutions can be made within the strand or can be made to residues at the ends of the strand. In some embodiments, the nucleotides mediate inhibition of translation of a target mRNA or are directed against the synthesis of a protein.

The target mRNA cleavage reaction guided by nucleotides of the present invention is sequence specific. In general, nucleotides containing a sequence identical to a portion of the target gene may be preferred for inhibition. However, 100% sequence identity between the nucleotide and the target gene is not required to practice the present invention. Sequence variations can be tolerated including those that might be expected due to genetic mutation, strain polymorphism, or evolutionary divergence. For example, nucleotide sequences with insertions, deletions, and single point mutations relative to the target sequence have also been found to be effective for inhibition. Alternatively, nucleotide sequences with nucleotide analog substitutions or insertions can be effective for inhibition. Accordingly, in one embodiment, the nucleotide of the present invention is identical to a reference nucleotide subsequence. In another embodiment, the nucleotide is exactly complementary to a reference nucleotide subsequence. In still another embodiment, the nucleotide is substantially complementary to a reference nucleotide subsequence.

Moreover, not all positions of a nucleotide contribute equally to target recognition. For example, mismatches in the center of an siRNA are most critical and essentially abolish target RNA cleavage. In contrast, the 3′ nucleotides of an siRNA do not contribute significantly to specificity of the target recognition. Generally, residues at the 3′ end of the siRNA sequence which is complementary to the target RNA (e.g., the guide sequence) are not critical for target RNA cleavage.

Sequence identity may readily be determined by sequence comparison and alignment algorithms known in the art. To determine the percent identity of two nucleic acid sequences (or of two amino acid sequences), the sequences are aligned for optimal comparison purposes (e.g., gaps can be introduced in the first sequence or second sequence for optimal alignment). The nucleotides (or amino acid residues) at corresponding nucleotide (or amino acid) positions are then compared. When a position in the first sequence is occupied by the same residue as the corresponding position in the second sequence, then the molecules are identical at that position. The percent identity between the two sequences is a function of the number of identical positions shared by the sequences (i.e., % homology=# of identical positions/total # of positions×100), optionally penalizing the score for the number of gaps introduced and/or length of gaps introduced.

The comparison of sequences and determination of percent identity between two sequences can be accomplished using a mathematical algorithm. In one embodiment, the alignment generated over a certain portion of the sequence aligned having sufficient identity but not over portions having low degree of identity (i.e., a local alignment). A preferred, non-limiting example of a local alignment algorithm utilized for the comparison of sequences is the algorithm of Karlin and Altschul (1990) Proc. NatL Acad. Sci. USA 87:2264-68, modified as in Karlin and Altschul (1993) Proc. NatL Acad. Sci. USA 90:5873. Such an algorithm is incorporated into the BLAST programs (version 2.0) of Altschul, et al. (1990) J Mol Biol. 215:403-10.

In another embodiment, the alignment is optimized by introducing appropriate gaps and percent identity is determined over the length of the aligned sequences (i.e., a gapped alignment). To obtain gapped alignments for comparison purposes, Gapped BLAST can be utilized as described in Altschul et al., (1997) Nucleic Acids Res. 25(17):3389. In another embodiment, the alignment is optimized by introducing appropriate gaps and percent identity is determined over the entire length of the sequences aligned (i.e., a global alignment). A preferred, non-limiting example of a mathematical algorithm utilized for the global comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

Greater than 90% sequence identity, e.g., 91%, 92%, 93%, 94%, 95%, 96%, 97%, 98%, 99% or even 100% sequence identity, between the nucleotide and the portion of the target mRNA is preferred. Alternatively, the nucleotide may be defined functionally as a nucleotide sequence (or oligonucleotide sequence) that is capable of hybridizing with a portion of the target mRNA transcript (e.g., 400 mM NaCl, 40 mM PIPES pH 6.4, 1 mM EDTA, 50° C. or 70° C. hybridization for 12-16 hours; followed by washing). Additional hybridization conditions include hybridization at 70° C. in 1×SSC or 50° C. in 1×SSC, 50% formamide followed by washing at 70° C. in 0.3×SSC or hybridization at 70° C. in 4×SSC or 50° C. in 4×SSC, 50% formamide followed by washing at 67° C. in 1×SSC. The hybridization temperature for hybrids anticipated to be less than 50 base pairs in length should be 5-10° C. less than the melting temperature (Tm) of the hybrid, where Tm is determined according to the following equations. For hybrids less than 18 base pairs in length, Tm(° C.)=2(# of A+T bases)+4(# of G+C bases). For hybrids between 18 and 49 base pairs in length, Tm(° C.)=81.5+16.6(log₁₀ [Na+])+0.41 (% G+C)−(600/N), where N is the number of bases in the hybrid, and [Na+] is the concentration of sodium ions in the hybridization buffer ([Na+] for 1×SSC=0.165 M). Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold 15 Spring Harbor, N.Y., chapters 9 and 11, and Current Protocols in Molecular Biology, 1995, F. M. Ausubel et al., eds., John Wiley & Sons, Inc., sections 2.10 and 6.3-6.4, incorporated herein by reference. The length of the identical nucleotide sequences may be at least about or about equal to 10, 12, 15, 17, 20, 22, 25, 27, 30, 32, 35, 37, 40, 42, 45, 47 or 50 bases.

In one embodiment, the nucleotides of the present invention are modified to improve stability in serum or in growth medium for cell cultures. In order to enhance the stability, the 3′-residues may be stabilized against degradation, e.g., they may be selected such that they consist of purine nucleotides, particularly adenosine or guanosine nucleotides. Alternatively, substitution of pyrimidine nucleotides by modified analogues, e.g., substitution of uridine by 2′-deoxythymidine is tolerated and does not affect the efficiency of RNA interference. For example, the absence of a 2′-hydroxyl may significantly enhance the nuclease resistance of the nucleotides in tissue culture medium.

In another embodiment of the present invention the nucleotide may contain at least one modified nucleotide analogue. The nucleotide analogues may be located at positions where the target-specific activity, e.g., the RNAi mediating activity is not substantially effected, e.g., in a region at the 5′-end and/or the 3′-end of the RNA molecule. Particularly, the ends may be stabilized by incorporating modified nucleotide analogues.

Nucleotide analogues include sugar- and/or backbone-modified ribonucleotides (i.e., include modifications to the phosphate-sugar backbone). For example, the phosphodiester linkages of natural RNA may be modified to include at least one of a nitrogen or sulfur heteroatom. In preferred backbone-modified ribonucleotides the phosphoester group connecting to adjacent ribonucleotides is replaced by a modified group, e.g., of phosphothioate group. In preferred sugar modified ribonucleotides, the 2′-OH-group is replaced by a group selected from OR, R, halo, SH, SR, NH₂, NHR, NR₂ or NO₂, wherein R is C1-C6 alkyl, alkenyl or alkynyl and halo is F, Cl, Br or I.

Nucleotide analogues also include nucleobase-modified ribonucleotides, L e., ribonucleotides, containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; O- and N-alkylated nucleotides, e.g., N6-methyl adenosine are suitable. It should be noted that the above modifications may be combined.

Nucleotides may be produced enzymatically or by partial/total organic synthesis and any modified ribonucleotide can be introduced by in vitro enzymatic or organic synthesis. In one embodiment, nucleotides of the present invention are prepared chemically. Methods of synthesizing RNA molecules are known in the art, in particular, the chemical synthesis methods as described in Verina and Eckstein (1998), Annual Rev. Biochem. 67:99. In another embodiment, nucleotides of the present invention are prepared enzymatically. For example, nucleotides can be prepared by enzymatic processing of a long, double-stranded RNA having sufficient complementarity to the desired target mRNA. Processing of long RNA can be accomplished in vitro, for example, using appropriate cellular lysates and siRNAs or morpholinos can be subsequently purified by gel electrophoresis or gel filtration. Nucleotides can then be denatured according to art-recognized methodologies. In an exemplary embodiment, nucleotides can be purified from a mixture by extraction with a solvent or resin, precipitation, electrophoresis, chromatography, or a combination thereof. Alternatively, the nucleotides may be used with no or a minimum of purification to avoid losses due to sample processing.

In one embodiment, nucleotides of the present invention are synthesized either in vivo, in situ, or in vitro. Endogenous RNA polymerase of the cell may mediate transcription in vivo or in situ, or cloned RNA polymerase can be used for transcription in vivo or in vivo. For transcription from a transgene in vivo or an expression construct, a regulatory region (e.g., promoter, enhancer, silencer, splice donor and acceptor, polyadenylation) may be used to transcribe the morpholinos. Inhibition may be targeted by specific transcription in an organ, tissue, or cell type; stimulation of an environmental condition (e.g., infection, stress, temperature, chemical inducers); and/or engineering transcription at a developmental stage or age. A transgenic organism that expresses a nucleotide of the present invention from a recombinant construct may be produced by introducing the construct into a zygote, an embryonic stem cell, or another multipotent cell derived from the appropriate organism.

Alternatively, nucleotides, e.g., siRNAs, can also be prepared by enzymatic transcription from synthetic DNA templates or from DNA plasmids isolated from recombinant bacteria. Typically, phage RNA polymerases are used such as T7, T3 or SP6 RNA polyimerase (Milligan and Uhlenbeck (1989) Methods Enzymol. 180:51-62). The RNA may be dried for storage or dissolved in an aqueous solution. The solution may contain buffers or salts to inhibit annealing, and/or promote stabilization of the double strands.

Commercially available design tools and kits, such as those available from Ambion, Inc. (Austin, Tex.), and the Whitehead Institute of Biomedical Research at MIT (Cambridge, Mass.) allow for the design and production of siRNA. By way of example, a desired mRNA sequence can be entered into a sequence program that will generate sense and antisense target strand sequences. These sequences can then be entered into a program that determines the sense and antisense siRNA oligonucleotide templates. The programs can also be used to add, e.g., hairpin inserts or T1 promoter primer sequences. Kits also can then be employed to build siRNA expression cassettes.

In some embodiments, the target mRNA expresses a protein which can be, but is not limited to a cancer protein, a virus protein, an HIV protein, a fungus protein, a bacterial protein, an abnormal cellular protein, and/or a normal cellular protein. For example, in one embodiment, the target mRNA of the invention specifies the amino acid sequence of at least one protein such as a cellular protein (e.g., a nuclear, cytoplasmic, transmembrane, or membrane-associated protein). In another embodiment, the target mRNA of the invention specifies the amino acid sequence of an extracellular protein (e.g., an extracellular matrix protein or secreted protein). As used herein, the phrase “specifies the amino acid sequence” of a protein means that the mRNA sequence is translated into the amino acid sequence according to the rules of the genetic code. The following classes of proteins are listed for illustrative purposes: developmental proteins (e.g., adhesion molecules, cyclin kinase inhibitors, Wnt family members, Pax family members, Winged helix family members, Hox family members, cytokines/lymphokines and their receptors, growth/differentiation factors and their receptors, neurotransmitters and their receptors); oncogene-encoded proteins (e.g., ABL1, BCL1, BCL2, BCL6, CBFA2. CBL, CSFIR, ERBA, ERBB, EBRB2, ETSI, ETS1, ETV6. FGR, FOS, FYN, HCR, HRAS, JUN, KRAS, LCK, LYN, MDM2, MLL, MYB, MYC, MYCLI, MYCN, NRAS, PIM 1, PML, RET, SRC, TALI, TCL3, and YES); tumor suppressor proteins (e.g., APC, BRCA1, BRCA2, MADH4, MCC, NF 1, NF2, RB 1, TP53, and WTI); and enzymes (e.g., ACC synthases and oxidases, ACP desaturases and hydroxylases, ADPglucose pyrophorylases, acetylases and deacetylases, ATPases, alcohol dehydrogenases, amylases, amyloglucosidases, catalases, cellulases, chalcone synthases, chitinases, cyclooxygenases, decarboxylases, dextrinases, DNA and RNA polymerases, galactosidases, glucanases, glucose oxidases, granule-bound starch synthases, GTPases, helicases, hemicellulases, integrases, inulinases, invertases, isomerases, kinases, lactases, lipases, lipoxygenases, lysozymes, nopaline synthases, octopine synthases, pectinesterases, peroxidases, phosphatases, phospholipases, phosphorylases, phytases, plant growth regulator synthases, polygalacturonases, proteinases and peptidases, pullanases, recombinases, reverse transcriptases, RUBISCOs, topoisomerases, and xylanases), proteins involved in tumor growth (including vascularization) or in metastatic activity or potential, including cell surface receptors and ligands as well as secreted proteins, cell cycle regulatory, gene regulatory, and apoptosis regulatory proteins, immune response, inflammation, complement, or clotting regulatory proteins.

As used herein, the term “oncogene” refers to a gene which stimulates cell growth and, when its level of expression in the cell is reduced, the rate of cell growth is reduced or the cell becomes quiescent. In the context of the present invention, oncogenes include intracellular proteins, as well as extracellular growth factors which may stimulate cell proliferation through autocrine or paracrine function. Examples of human oncogenes against which nucleotide constructs, e.g., siRNA or morpholino constructs can be designed include c-myc, c-myb, mdm2, PKA-I (protein kinase A type I), Abl-1, Bcl2, Ras, c-Raf kinase, CDC25 phosphatases, cyclins, cyclin dependent kinases (cdks), telomerase, PDGF/sis, erb-B, fos, jun, mos, and src, to name but a few. In the context of the present invention, oncogenes also include a fusion gene resulted from chromosomal translocation, for example, the Bcr/Abl fusion oncogene.

Further proteins include cyclin dependent kinases, c-myb, c-myc, proliferating cell nuclear antigen (PCNA), transforming growth factor-beta (TGF-beta), and transcription factors nuclear factor kappaB (NF-.kappa.B), E2F, HER-2/neu, PKA, TGF-alpha, EGFR, TGF-beta, IGFIR, P12, MDM2, BRCA, Bcl-2, VEGF, MDR, ferritin, transferrin receptor, IRE, C-fos, HSP27, C-raf and metallothionein genes.

The nucleotides employed in the present invention can be directed against the synthesis of one or more proteins. Additionally or alternatively, there can be more than one nucleotide directed against a protein, e.g., duplicate nucleotides or nucleotides that correspond to overlapping or non-overlapping target sequences against the same target protein. Additionally, several nucleotides directed against several proteins can be employed. Accordingly, in one embodiment two, three, four or any plurality of nucleotides against the same target mRNA can be including in the cochleate compositions of the invention. Alternatively, the nucleotides can be directed against structural or regulatory RNA molecules that do not code for proteins.

In a preferred aspect of the invention, the target mRNA molecule of the invention specifies the amino acid sequence of a protein associated with a pathological condition. For example, the protein may be a pathogen-associated protein (e.g., a viral protein involved in immunosuppression or immunoavoidance of the host, replication of the pathogen, transmission of the pathogen, or maintenance of the infection), or a host protein which facilitates entry of the pathogen into the host, drug metabolism by the pathogen or host, replication or integration of the pathogen's genome, establishment or spread of infection in the host, or assembly of the next generation of pathogen. Alternatively, the protein may be a tumor-associated protein or an autoimmune disease-associated protein.

In one embodiment, the target mRNA molecule of the invention specifies the amino acid sequence of an endogenous protein (i.e. a protein present in the genome of a cell or organism). In another embodiment, the target mRNA molecule of the invention specifies the amino acid sequence of a heterologous protein expressed in a recombinant cell or a genetically altered organism. In another embodiment, the target mRNA molecule of the invention specifies the amino acid sequence of a protein encoded by a transgene (i.e., a gene construct inserted at an ectopic site in the genome of the cell). In yet another embodiment, the target mRNA molecule of the invention specifies the amino acid sequence of a protein encoded by a pathogen genome which is capable of infecting a cell or an organism from which the cell is derived.

By inhibiting the expression of such proteins, valuable information regarding the function of said proteins and therapeutic benefits which may be obtained from said inhibition may be obtained.

Accordingly, in one embodiment, the nucleotide-cochleate compositions of the present invention can be utilized in studies of mammalian cells to clarify the role of specific structural and catalytic proteins. In another embodiment, they can be used in a therapeutic application to specifically target pathogenic organisms, including fungi, bacteria, and viruses.

Cochleates and methods for making and using have been disclosed, e.g., in U.S. Pat. Nos. 5,999,318 and 6,592,894. Cochleate delivery vehicles are stable lipid-cation precipitates that can be composed of simple, naturally occurring materials, e.g., phosphatidylserine, and calcium. Mixtures of naturally occurring molecules (e.g., soy lipids) and/or synthetic or modified lipids can be utilized.

The cochleate structure provides protection from degradation for associated “encochleated” moieties. Divalent cation concentrations in vivo in serum and mucosal secretions are such that the cochleate structure is maintained. Hence, the majority of cochleate-associated molecules, e.g., cargo moieties, are present in the inner layers of a primarily solid, non-aqueous, stable, impermeable structure. Since the cochleate structure includes a series of solid layers, components within the interior of the cochleate structure remain substantially intact, even though the outer layers of the cochleate may be exposed to harsh environmental conditions or enzymes.

The cochleate interior is primarily free of water and resistant to penetration by oxygen. Oxygen and water are primarily responsible for the decomposition and degradation of molecules which can lead to reduced shelf-life. Accordingly, encochleation should also impart extensive shelf-life stability to encochleated nucleotides.

With respect to storage, cochleates can be stored in cation-containing buffer, or lyophilized or otherwise converted to a powder, and stored at room temperature. If desired, the cochleates also can be reconstituted with liquid prior to administration. Cochleate preparations have been shown to be stable for more than two years at 4° C. in a cation-containing buffer, and at least one year as a lyophilized powder at room temperature.

In one embodiment, the cochleate comprises a negatively charged lipid component and a multivalent cation component. The lipid employed in the present invention may include one or more negatively charged lipids. As used herein, the term “negatively charged lipid” includes lipids having a head group bearing a formal negative charge in aqueous solution at an acidic, basic or physiological pH, and also includes lipids having a zwitterionic head group.

In one embodiment, the lipid is a mixture of lipids, comprising at least 75% negatively charged lipid. In another embodiment, the lipid includes at least 85% negatively charged lipid. In other embodiments, the lipid includes at least 90%, 95% or even 99% negatively charged lipid. All ranges and values between 60% and 100% negatively charged lipid are meant to be encompassed herein.

The negatively charged lipid can include soy-based lipids. Preferably, the lipid includes phospholipids, such as soy phospholipids (soy-based phospholipids). The negatively charged lipid can include phosphotidyl serine (PS), dioleoylphosphatidylserine (DOPS), phosphatidic acid (PA), phosphatidylinositol (PI), and/or phosphatidyl glycerol (PG) and or a mixture of one or more of these lipids with other lipids. Additionally or alternatively, the lipid can include phosphatidylcholine (PC), phosphatidylethanolamine (PE), diphosphotidylglycerol (DPG), dioleoyl phosphatidic acid (DOPA), distearoyl phosphatidylserine (DSPS), dimyristoyl phosphatidylserine (DMPS), dipalmitoyl phosphatidylgycerol (DPPG) and the like.

The lipids can be natural or synthetic. For example, the lipid can include esterified fatty acid acyl chains, or organic chains attached by non-ester linkages such as ether linkages (as described in U.S. Pat. No. 5,956,159), disulfide linkages, and their analogs.

In one embodiment the lipid chains are from about 6 to about 26 carbon atoms, and the lipid chains can be saturated or unsaturated. Fatty acyl lipid chains useful in the present invention include, but are not limited to, n-tetradecanoic, n-hexadecanoic acid, n-octadecanoic acid, n-eicosanoic acid, n-docosanoic acid, n-tetracosanoic acid, n-hexacosanoic acid, cis-9-hexadecenoic acid, cis-9-octadecenoic acid, cis,cis-9,12-octadecedienoic acid, all-cis-9,12,15-octadecetrienoic acid, all-cis-5,8,11,14-eicosatetraenoic acid, all-cis-4,7,10,13,16,19-docosahexaenoic acid, 2,4,6,8-tetramethyl decanoic acid, and lactobacillic acid, and the like.

In some embodiments, pegylated lipid also is included. Pegylated lipid includes lipids covalently linked to polymers of polyethylene glycol (PEG). PEG's are conventionally classified by their molecular weight, thus PEG 6,000 MW, e.g., has a molecular weight of about 6000. Adding pegylated lipid generally will result in an increase of the amount of compound (e.g., peptide, nucleotide, and nutrient) that can be incorporated into the precipitate. An exemplary pegylated lipid is dipalmitoylphosphatidylehtanolamine (DPPE) bearing PEG 5,000 MW. In some embodiments, a portion of the lipid is not pegylated. In other embodiments, none of the lipid used to make the cochleate is pegylated.

The nucleotide-cochleate compositions of the present invention can be provided in a variety of forms (e.g. powder, liquid, suspension) with or without additional components. Suitable forms and additives, excipients, carriers and the like are known in the art.

The cochleates and cochleate compositions of the present invention can optionally include an aggregation inhibitor. Aggregation inhibitors work in part by modifying the surface characteristics of the cochleates such that aggregation is inhibited. Aggregation can be inhibited, for example, by steric bulk and/or a change in the nature of the cochleate structure, e.g., a change in the surface hydrophobicity and/or surface charge.

Aggregation can be inhibited by including in the liposome suspension a material that prevents liposome-liposome interaction at the time of calcium addition and thereafter. Alternatively, the aggregation inhibitor can be added after formation of cochleates. Additionally, the amount of aggregation inhibitor can be varied, thus allowing modulation of the size of the cochleates. The use of aggregation inhibitors in cochleate compositions can be found, e.g., in WO 04/091578.

The cochleates and cochleate compositions of the present invention can further include one or more additional cargo moieties. An “additional cargo moiety” is an encochleated moiety in addition to the nucleotide of the invention, and generally does not refer to the lipid and ion employed to precipitate the cochleate. Cargo moieties include any compounds having a property of biological interest, e.g., ones that have a role in the life processes of a living organism. A cargo moiety may be organic or inorganic, a monomer or a polymer, endogenous to a host organism or not, naturally occurring or synthesized in vitro and the like.

Exemplary additional cargo moieties are disclosed in WO 04/091578, the entire contents of which are incorporated by this reference. Cargo moieties include, but are not limited to, vitamins, minerals, nutrients, micronutrients, amino acids, toxins, microbicides, microbistats, co-factors, enzymes, polypeptides, polypeptide aggregates, polynucleotides, lipids, carbohydrates, nucleotides, starches, pigments, fatty acids, monounsaturated fatty acids, polyunsaturated fatty acids, flavorings, essential oils, extracts, hormones, cytokines, viruses, organelles, steroids and other multi-ring structures, saccharides, metals, metabolic poisons, imaging agents, antigens, porphyrins, tetrapyrrolic pigments, marker compounds, medicaments, drugs and the like.

### Methods of Manufacture

In another aspect, the present invention generally is directed to methods of making cochleates that include the nucleotides described herein, e.g., those associated with a transfection agent or bound to a cochleate component or lipid tail. The methods generally can include precipitating a liposome suspension in the presence of a nucleotide component, e.g., by adding a multivalent cation. The cochleates can further include additional cargo moieties or other constituents, e.g., aggregation inhibitors. All of the methods described herein can be employed for making nucleotide-cochleates.

Liposomes may be prepared by any known method of preparing liposomes. Thus, the liposomes may be prepared for example by solvent injection, lipid hydration, reverse evaporation, freeze drying by repeated freezing and thawing. The liposomes may be multilamellar or unilamellar, including small unilamellar vesicles (SUV). The liposomes may be large unilamellar vesicles (LUV), stable plurilamellar vesicles (SPLV) or oligolamellar vesicles (OLV) prepared, e.g., by detergent removal using dialysis, column chromatography, bio beads SM-2, by reverse phase evaporation (REV), or by formation of intermediate size unilamellar vesicles by high pressure extrusion. Methods in Biochemical Analysis, 33:337 (1988). Liposomes made by all these and other methods known in the art can be used in practicing this invention.

In a preferred embodiment at least majority of the liposomes are unilamellar. The method can further include the step of filtering a liposomal suspension and/or mechanically extruding the suspension through a small aperture that includes both MLV and ULV liposomes, such that a majority of the liposomes are ULV. In preferred embodiments, at least 70%, 80%, 90% or 95% of the liposomes are ULV.

The method is not limited by the method of forming cochleates. Any known method can be used to form cochleates from the liposomes of the invention (i.e., the liposomes associated with the nucleotide). In one embodiment, known methods can be employed to form the cochleates of the invention, including but not limited to those described in U.S. Pat. Nos. 5,994,318 and 6,153,217, the entire disclosures of which are incorporated herein by this reference.

In one embodiment, prior to precipitation, SUVs are obtained by, e.g., filtration, and the liposomes are precipitated in the presence of nucleotides and/or other cargo moiety to form cochleates.

In another embodiment, MLVs are extruded one or more times in the presence of nucleotides and/or other cargo moiety, then the liposomes are precipitated form cochleates. In this embodiment, it is believed that the MLVs open and reseal during the extrusion process thereby entrapping or otherwise increasing the association of the nucleotides and/or other cargo moieties with the MLVs.

In yet another embodiment, a chelating agent (e.g., EDTA) is employed to convert cochleates to liposomes in the presence of the nucleotides and/or other cargo moiety, and then cation is added to form the cochleates.

Additionally or alternatively, nucleotides can be encochleated with high or low amounts of calcium. Accordingly, in one embodiment, a high or “elevated” amount of calcium is used, e.g., wherein the calcium concentration in the solution when the cochleates are formed is between about 100 and about 500 mM. As used herein, the term “elevated amount of calcium” means a calcium concentration between about 100 and about 500 mM. In another embodiment, a relatively low (“depressed”) amount of calcium is used, e.g., between about 1 to about 10 mM. As used herein, the term “depressed amount of calcium” means a calcium concentration between about 1 and about 10 mM. In some cases, siRNA encochleated with high amounts of calcium were more active than siRNA encochleated with low amounts of calcium. Accordingly, it is believed that the use of cochleates of the invention made with higher calcium concentrations may result in more active nucleotides generally.

In one embodiment, the pH of the nucleotide is adjusted in order to induce a charge in the molecule and thereby increase its interaction with the cochleate, and in particular the phospholipid. In one embodiment, the method includes adjusting the pH of the liposomal suspension. In another embodiment, the method may include charging the base pairs of the nucleotide. For example, the pH can be adjusted to about 8.5 or about 6.0 to 6.5 or about 3.0 to 3.5 for a morpholino. Raising the pH of a liposomal suspension in the presence of morpholino causes the morpholino to associate or complex with the liposomes. Raising or lowering the pH of the nucleotide (between 3 to 11) can affect charge on the bases or backbone and enhance association with the lipid.

It has been discovered that adjusting the pH and/or charging the base pairs can improve association of the nucleotide with the cochleate. Accordingly, the method can further include the step of adjusting the pH of the nucleotide prior to or during the contact with the liposome or formation of the precipitate. Any known method of adjusting pH can be employed. For example, a nucleotide can be acidified with acidic aqueous buffer. Alternately, pH can be raised with a basic aqueous buffer. Acidic and basic buffers are known in the art, and identification of a variety of buffers would require no more than routine experimentation by one of ordinary skill in the art. Alternatively, the pH of the nucleotide can be adjusted by slow addition of an acid, e.g., hydrochloric acid, or a base, e.g., sodium hydroxide.

In yet other embodiments, the pH of the nucleotide can be adjusted prior to incorporation into the lipid precipitates. In other embodiments, the pH of the resultant a nucleotide-cochleates in solution can be adjusted using, e.g., acid or base.

In one embodiment, cochleates may be formed by dissolving a lipid component and a nucleotide and/or other cargo moiety in an organic solvent (e.g., THF) to form a solution, forming nucleotide liposomes, and precipitating the liposomes to form a nucleotide-cochleate. The solvent can optionally be removed prior to the formation of liposomes and/or after the liposomes are formed.

In another embodiment, cochleates can be formed by introducing the molecule (e.g, nucleotide and/or additional cargo moiety), to a liposome in the presence of a solvent such that the molecule associate with the liposome, and precipitating the liposome to form a cochleate. The molecule can be introduced by introducing a solution of the solvent and the molecule to the liposome by, e.g., dropwise addition, continuous flow or as a bolus. The molecule can also be introduced to the liposome prior to or after the solvent.

The liposome may be prepared by any known method of preparing liposomes. Additionally, the method is not limited by the method of forming cochleates. Any known method can be used to form cochleates from the liposomes of the invention (i.e., the liposomes associated with the cargo moiety). In a preferred embodiment, the cochleate is formed by precipitation. Additionally or alternatively, an aggregation inhibitor can be added to the solvent at the liposomal stage, or to the precipitated cochleate.

Any suitable solvent can be employed in connection with the present invention. Solvents suitable for a given application can be readily identified by a person of skill in the art. Suitable solvents include but are not limited to dimethylsulfoxide (DMSO), a methylpyrrolidone, N-methylpyrrolidone (NMP), acetonitrile, alcohols, e.g., butanol and ethanol (EtOH), dimethylformamide (DMF), tetrahydrofuran (THF), and combinations thereof.

Moreover, the order of addition of various components (e.g., nucleotide, lipid, calcium, cation complexing agents, solvent) can readily be varied. Concentrations and ratios of various components can also be modified as exemplified herein. Finally, ionic conditions may be adjusted as appropriate. Salt concentrations may be approximately isotonic (150 mM), to high (e.g., 1 to 2 molar), to hypotonic, to zero (water).

An exemplary method of forming nucleotide-cochleates in accordance with the present invention can generally include the following steps. Liposomes and nucleotides can be solubilized and vortexed to form a nucleotide-liposome suspension. Typically, about 2 minutes of vortexing is sufficient to form a suitable suspension, which can be varied and confirmed by visual inspection, e.g., through a microscope.

Next, the pH of the suspension is either raised to about 8.5 (e.g., with 1 N NaOH) or lowered to about 6.5 (e.g., with 1 N HCl). Since some nucleotides, e.g., morpholinos, are non-charged, this step may done to place a charge on the base pairs, to favor an interaction with the liposomes. This ionic interaction can be achieved by either increasing the pH to 8.5 or by lowering the pH to 6.5. At this point the nucleotides interact with the lipid. The suspension is again vortexed to induce interaction between the nucleotides and the liposomes. Typically, about 10 minutes of vortexing is suitable. Interaction between the nucleotides and the liposomes can be confirmed by phase and diffraction microscopy. The nucleotides associate with or incorporate into the liposomal bilayer. The nucleotide-liposomes are then filtered (e.g., using a 0.22 micrometer syringe filter).

Calcium solution is added to the suspension with vortexing. A suitable addition technique is to use an eppendorf repeater pipetter with a 500 microliter tip, and to add 10 microliter aliquots to the tube every 10 seconds until cochleates are formed. Cochleate formation can be confirmed, e.g., by observing the preparation under a microscope and by a measurement of pH. The cochleates can then be stably stored at 4° C. in a nitrogen atmosphere.

### Methods of Use

In another aspect, the invention provides methods of administering any of the compositions described herein to a host (e.g., a cell or organism). The method generally includes administering a biologically effective amount of a nucleotide-cochleate composition to a host. The cochleate compositions can include any of the compositions described herein including, e.g., compositions with additional cargo moieties and/or aggregation inhibitors.

The host can be a cell, a cell culture, an organ, a tissue, and organism, an animal etc. For example, in one embodiment, the nucleotide is delivered to a cell in the host (e.g., to a cytosol compartment of the cell).

In one embodiment the nucleotide mediates RNAi against a target mRNA in the host. In another embodiments, the nucleotide mediates translation of a target mRNA in the host. In either embodiment, although acting by a different mechanism, specific target protein synthesis preferably is reduced in the host. In preferred embodiments, target protein synthesis is reduced by at least about 10%, 20%, 30%, 40%, 50%, 60%, 70%, 80%, 90%, or even 100%.

Physical methods of introducing nucleotides to cells and organisms employing cochleates include contacting the cells with the cochleates or administering the cochleates to the organism by any means, e.g., orally, intramuscularly, intradermally, transdermally, intranasally, intrarectally, subcutaneously, topically, or intravenously. Nucleotide-cochleates may be introduced to or into a call using a number of mechanisms, methods, or routes, all of which are known in the art. See, e.g., WO 04/091572.

A cell or tissue with a target mRNA may be derived from or contained in any organism. The organism may be a plant, animal, protozoan, bacterium, virus, or fungus, as also described in, e.g., WO 04/091572. The cell having the target mRNA may be from the germ line or somatic, totipotent or pluripotent, dividing or non-dividing, parenchyma or epithelium, immortalized or transformed, or the like. The cell may be a stem cell or a differentiated cell. Cell types that are differentiated include adipocytes, fibroblasts, myocytes, cardiomyocytes, endothelium, neurons, glia, blood cells, megakaryocytes, lymphocytes, macrophages, neutrophils, eosinophils, basophils, mast cells, leukocytes, granulocytes, keratinocytes, chondrocytes, osteoblasts, osteoclasts, hepatocytes, and cells of the endocrine or exocrine glands.

Depending on the particular target mRNA and the dose of nucleotide material delivered, this process may provide partial or complete loss of function for the target mRNA in a host. A reduction or loss of mRNA expression in at least 20%, 30%, 40%, 50%, 60%, 70%, 80%, 90%, 95% or 99% or more of the host or targeted cells in the host is exemplary. Inhibition of mRNA expression refers to the absence (or observable decrease) in the level of protein and/or mRNA product from a target mRNA. Specificity refers to the ability to inhibit the target mRNA without manifest effects on other genes of the cell. The consequences of inhibition can be confirmed by examination of the outward properties of the cell or organism or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). Assays for assessing delivery and activity of the compositions of the present invention, as well as assays for mRNA expression are described, e.g., in WO 04/091572

The nucleotide may be introduced in an amount which allows delivery of at least one copy per cell. Higher doses (e.g., at least 5, 10, 100, 500 or 1000 copies per cell) of material may yield more effective inhibition; lower doses may also be useful for specific applications.

The cochleates can be coadministered with a further agent. The second agent can be delivered in the same cochleate preparation, in a separate cochleate preparation mixed with the cochleates preparation of the invention, separately in another form (e.g., capsules or pills), or in a carrier with the cochleate preparation. The cochleates can further include one or more additional cargo moieties, such as other drugs, peptides, nucleotides (e.g., DNA and RNA), antigens, nutrients, flavors and/or proteins. Such molecules have been described in U.S. Pat. Nos. 6,153,217 (Jin et al.) and 5,994,318 (Gould-Fogerite et al.), and International Patent Publication Nos. WO 00/42989 (Zarif et al.) and WO 01/52817 (Zarif et al.). These patents are expressly incorporated by this reference.

The cochleates of the invention also can include a reporter molecule for use in in vitro diagnostic assays, which can be a fluorophore, radiolabel or imaging agent. The cochleates can include molecules that direct binding of the cochleate to a specific cellular target, or promotes selective entry into a particular cell type.

Another advantage of the present invention is the ability to modulate cochleate size. Modulation of the size of cochleates can change the manner in which the nucleotide and/or additional cargo moiety is taken up by cells. For example, in general, small cochleates are taken up quickly and efficiently into cells, whereas larger cochleates are taken up more slowly, but tend to retain efficacy for a longer period of time. Also, in some cases small cochleates are more effective than large cochleates in certain cells, while in other cells large cochleates are more effective than small cochleates.

### Methods of Treatment

In another aspect, the present invention provides for both prophylactic and therapeutic methods of treating a subject at risk of (or susceptible to) a disorder or having a disorder associated with aberrant or unwanted target gene expression or activity. The method generally includes administering to a subject a therapeutically effective amount of a nucleotide-cochleate of the invention such that the disease or disorder is treated.

The present invention provides a method for treating a subject that would benefit from administration of a composition of the present invention. Any therapeutic indication that would benefit from the cochleate compositions of the present invention can be treated by the methods of the invention. The method includes the step of administering to the subject a composition of the invention, such that the disease or disorder is treated.

Methods of treatment (prophylactic and therapeutic), including therapeutically effective amounts, are described, e.g., in WO 04/091572.

One advantage of the cochleates of the present invention is the safety and stability of the composition. Cochleates can be administered orally or by instillation without concern, as well as by the more traditional routes, such as oral, intranasal, intraoculate, intrarectal, intravaginal, intrapulmonary, topical, subcutaneous, intradermal, intramuscular, intravenous, subcutaneous, transdermal, systemic, intrathecal (into CSF), and the like. Direct application to mucosal surfaces is an attractive delivery means made possible with cochleates.

The disease or disorder treated in accordance with the present invention can be any disease or disorder that can be treated by the successful administration of the nucleotides of the invention. Exemplary diseases and disorders include neurological disorders associated with aberrant or unwanted gene expression such as schizophrenia, obsessive compulsive disorder (OCD), depression and bipolar disorder, Alzheimer's disease, Parkinson's disease, lymphoma, immune-mediated inflammatory disorders, hyperplasia, cancers, cell proliferative disorders, blood coagulation disorders, Dysfibrinogenaemia and hemophelia (A and B), dermatological disorders, hyperlipidemia, hyperglycemia, hypercholesterolemia, obesity, acute and chronic leukemias and lymphomas, sarcomas, adenomas, fungal infections, bacterial infections, viral infections, a lysosomal storage disease, Fabry's disease, Gaucher's Disease, Type I Gaucher's Disease, Farber's disease, Niemann-Pick disease (types A and B), globoid cell leukodystrophy (Krabbe's disease), metachromic leukodystrophy, multiple sulfatase deficiency, sulfatidase activator (sap-B) deficiency, sap-C deficiency, G_{M1}-gangliosidosis, Tay-Sachs disease, Tay-Sachs B1 variant, Tay-Sachs AB variant, Acid Maltase Deficiency, Mucopolysaccharidosis, Sandhoff's disease, a cancer, an autoimmune disorder, systemic lupus erythematosis, multiple sclerosis, myasthenia gravis, autoimmune hemolytic anemia, autoimmune thrombocytopenia, Grave's disease, allogenic transplant rejection, rheumatoid arthritis, ankylosing spondylitis, psoriasis, scleroderma, carcinomas, epithelial cancers, small cell lung cancer, non-small cell lung cancer, prostate cancer, breast cancer, pancreatic cancer, hepatocellular carcinoma, renal cell carcinoma, biliary cancer, colorectal cancer, ovarian cancer, uterine cancer, melanoma, cervical cancer, testicular cancer, esophageal cancer, gastric cancer, mesothelioma, glioma, glioblastoma, pituitary adenomas, inflammatory diseases, osteoarthritis, atherosclerosis, inflammatory bowel diseases (Crohns and ulcerative colitis), uveitis, eczema, chronic rhinosinusitis, asthma, a hereditary disease, cystic fibrosis, and muscular dystrophy.

The method can also be used for regulating gene expression to promote greater health or quality of life, e.g., to limit cholesterol uptake or regulate lipid metabolism, weight gain, hunger, aging, or growth. Cosmetic effects such as wrinkle reduction, hair growth, pigmentation, or dermatologic disorders may also be treated.

The compositions of the present invention can be used to enhance antiviral defense, transposon silencing, gene regulation, centromeric silencing, and genomic rearrangements. The compositions of the invention can also be used to inhibit expression of other types of RNA, e.g., ribosomal RNA, transfer RNA, and small nuclear RNA.

The nucleotide cochleate compositions of the present invention can be utilized in any number of gene therapies. One such treatment is for the management of opportunistic fungal infections like Aspergillus fumigatus, particularly in immunocompromised patients. Current treatment protocols with existing antifungal agents can still result in mortality rates of 80% in HIV patients or those undergoing cancer-related chemotherapies. However, the targeted disruption of the P-type H+-ATPase, an important plasma membrane enzyme critical to fungal cell physiology, may be an alternate and more effective way to destroy fungi such as A. fumigatus. This particular ATPase was cloned and selective small interfering RNA (siRNA) oligonucleotides obtained, which can knockdown the expression of this critical protein, resulting in the death of the fungus.

The essential role of the H+-ATPase in spore germination and multiplication of growing cells provides an opportunity to explore the ability of nanocochleates to efficiently deliver siRNAs targeted to the H+-ATPase of A. fumigatus. Given the medical importance of A. fumigatus and the paucity of available antifungal compounds, compositions of the present invention, e.g., siRNA cochleate compositions have the potential to be effective therapeutic alternatives.

### Combination Therapies

The above methods can be employed in the absence of other treatment, or in combination with other treatments. Such treatments can be started prior to, concurrent with, or after the administration of the compositions of the instant invention. Accordingly, the methods of the invention can further include the step of administering a second treatment, such as a second treatment for the disease or disorder or to ameliorate side effects of other treatments. Such second treatment can include, e.g., any treatment directed toward reducing an immune response. Additionally or alternatively, further treatment can include administration of drugs to further treat the disease or to treat a side effect of the disease or other treatments (e.g., anti-nausea drugs).

In one aspect, the invention provides a method for preventing in a subject, a disease or disorder which can be treated with administration of the compositions of the invention. Subjects at risk for a disease or condition which can be treated with the agents mentioned herein can be identified by, for example, any or a combination of diagnostic or prognostic assays known to those skilled in the art. Administration of a prophylactic agent can occur prior to the manifestation of symptoms characteristic of the disease or disorder, such that the disease or disorder is prevented or, alternatively, delayed in its progression.

### Pharmaceutical Compositions

The invention pertains to uses of the cochleates of the invention for prophylactic and therapeutic treatments as described infra. Accordingly, the cochleates of the present invention can be incorporated into pharmaceutical compositions suitable for administration. Such compositions typically comprise the cochleates of the invention and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art.

Cochleates of the present invention readily can be prepared from safe, simple, well-defined, naturally occurring substances, e.g., phosphatidylserine (PS) and calcium. Phosphatidylserine is a natural component of all biological membranes, and is most concentrated in the brain. The phospholipids used can be produced synthetically, or prepared from natural sources. Soy PS is inexpensive, available in large quantities and suitable for use in humans. Additionally, clinical studies indicate that PS is safe and may play a role in the support of mental functions in the aging brain. Unlike many cationic lipids, cochleates (which are composed of anionic lipids) are non-inflammatory and biodegradable. The tolerance in vivo of mice to multiple administrations of cochleates by various routes, including intravenous, intraperitoneal, intranasal and oral, has been evaluated. Multiple administrations of high doses of cochleate compositions to the same animal show no toxicity, and do not result in either the development of an immune response to the cochleate matrix, or any side effects relating to the cochleate vehicle.

The cochleates of the present invention can be administered to animals, including both human and non-human animals. It can be administered to animals, e.g., in animal feed or water. Methods for preparing pharmaceutical compositions containing the compositions of the present invention, including additional agents (e.g., wetting agents, emulsifiers and lubricants) used in such compositions, may be dependent upon the method of administration. Such method are known in the art, e.g., in WO 04/091572.

In some embodiments, adjuvants or immunomodulators may be added to the compositions of the present invention to stimulate an immune response. The immunomodulator can include comprise envelope proteins derived from human or animal viruses, oligonucleotides, e.g., CpG oligonucleotides, or can be chemical in nature. Specific chemical immunomodulators include, but are not limited to cytokines, chemokines and lymphokines, including, but not limited to, interferon alpha, interferon gamma, and interleuken 12. Examples of suitable animal viruses as a source of envelope protein include, but are not limited to, viruses from the following families: Arenaviridae, Bunyaviridae, Coronaviridae, Deltaviridae, Flaviviridae, Herpesviridae, Rhabdoviridae , Retroviridae, Poxyiridae, Paramyxoviridae, Orthomyxoviridae, and Togaviridae. Envelope proteins from influenza virus, Newcastle disease virus, and vaccinia virus, and Sendai virus are also encompassed in the present invention.

The pharmaceutical compositions can be included in a container, pack, or dispenser together with instructions for administration. The pharmaceutical compositions can be included in a container along with one or more additional compounds or compositions and instructions for use. For example, the invention also provides for packaged pharmaceutical products containing two agents, each of which exerts a therapeutic effect when administered to a subject in need thereof. A pharmaceutical composition may also comprise a third agent, or even more agents yet, wherein the third (and fourth, etc.) agent can be another agent against the disorder, such as a cancer treatment (e.g., an anticancer drug and/or chemotherapy) or an HIV cocktail. In some cases, the individual agents may be packaged in separate containers for sale or delivery to the consumer. The agents of the invention may be supplied in a solution with an appropriate solvent or in a solvent-free form (e.g., lyophilized). Additional components may include acids, bases, buffering agents, inorganic salts, solvents, antioxidants, preservatives, or metal chelators. The additional kit components are present as pure compositions, or as aqueous or organic solutions that incorporate one or more additional kit components. Any or all of the kit components optionally further comprise buffers.

The present invention also includes packaged pharmaceutical products containing a first agent in combination with (e.g., intermixed with) a second agent. The invention also includes a pharmaceutical product comprising a first agent packaged with instructions for using the first agent in the presence of a second agent or instructions for use of the first agent in a method of the invention. The invention also includes a pharmaceutical product comprising a second or additional agents packaged with instructions for using the second or additional agents in the presence of a first agent or instructions for use of the second or additional agents in a method of the invention. Alternatively, the packaged pharmaceutical product may contain at least one of the agents and the product may be promoted for use with a second agent.

The present invention further includes the methods and compositions described in U.S. Ser. No. 10/822,230, filed Apr. 9, 2004, and any combinations thereof with the methods and compositions described herein.

### EXEMPLIFICATION

### Example 1

### Cochleates Prepared with siRNA-PEI Complexes

siRNA and polyethylenimine (PEI) were allowed to associate to form a positively charged complex and then bound to negatively charged liposomes and encochleated. The effect of these encochleated complexes was studied.

22.5 μl of siRNA (20 μM) was added to an Eppendorf micro-centrifuge tube. 16.2 μA of PEI (2000 MW, Lupasol G35, BASF) at a concentration of 0.05%, was added and mixed well. Then, 116 μl of pre-made DOPS liposome at 1.5 mg/ml (in TES, pH7.0) was added to this mixture and mixed well. Finally, 115 μl of 0.1 M calcium chloride was added and mixed well to form cochleates. Cochleate morphology was confirmed microscopically. In order remove any free (unencochleated) siRNA from the siRNA-cochleate composition, the siRNA-cochleates were pelleted by centrifugation and the supernatants removed. Pellets were re-suspended.

Cochleates also were formed with non-specific siRNA (no specificity against erbB2 and no known intracellular target) according to the same method. The anti Erb siRNA-cochleates and non-specific siRNA-cochleates (both formed with PEI) were administered to SKOV3 cells at 0.25 μg (full dose) and 0.125 μg (50% dose), alongside untreated SKOV3 cells and were incubated for 72 hours.

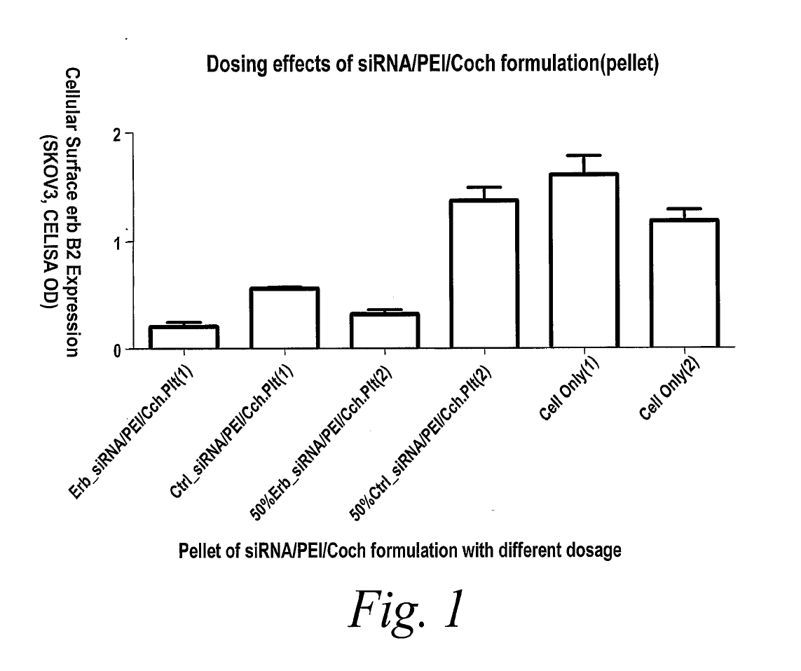

As summarized in FIG. 1, SKOV3 cells treated with the siRNA/PEI-cochleate compositions (Erb_siRNA/PEI/Cch.Plt (1)), showed a significant reduction in Erb B staining compared to untreated cells (Cell only (1)). Analogous compositions with a non-specific siRNA showed statistically less inhibition (CtrlErb_siRNA/PEI/Cch.Plt (1)). When half the concentration of siRNA/PEI-cochleates were used, the anti-Erb B siRNA/PEI-cochleates (ErbB Plt(2)) continued to cause a significant reduction in Erb B staining, but the control cochleates (Ctl.Plt(2)) showed no inhibition compared to untreated cells (Cell Only (2)). This indicates an anti-ErbB2-specific effect of the cochleate delivered siRNA.

The siRNA/PEI-cochleates were compared to SKOV3 cells treated with (1) unencochleated siRNA/PEI complex, (2) encochleated Fetal Bovine Serum (FBS) and PEI, (3) unencochleated FBS and PEI, and untreated cells. These controls were formulated by the same methods and in the same quantities and concentrations as the siRNA cochleates.

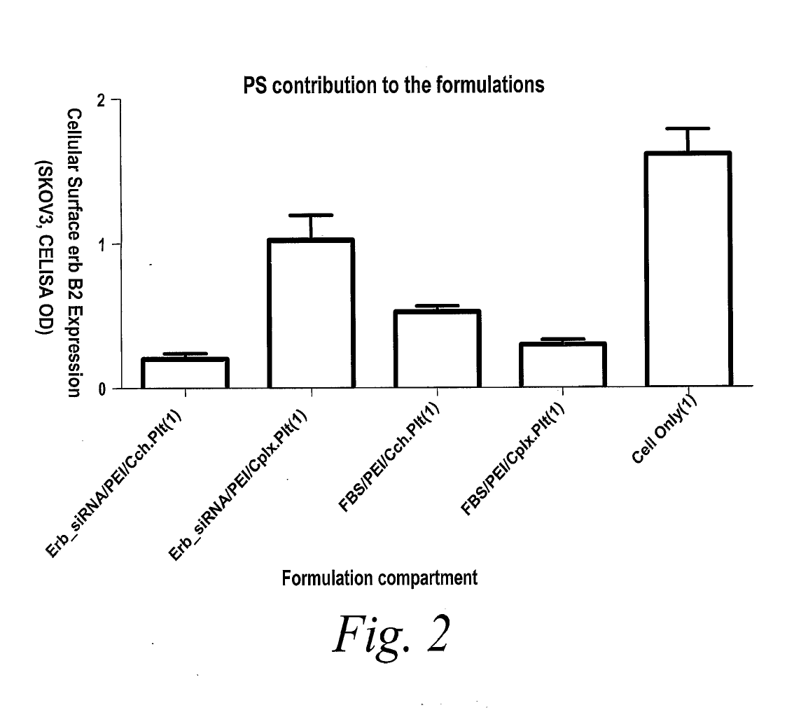

As summarized in FIG. 2, greater inhibition of Erb B was seen upon administration of siRNA/PEI-cochleates (Erb_siRNA/PEI/Cch.Plt(1)), as compared to the unencochleated siRNA/PEI (Erb_siRNA/PEI/Cplz.Plt(1)), indicating a positive role for cochleate delivery of siRNA. FBS/PEI-cochleates (FBS/PEI/Cch.Plt(1)), and unencochleated FBS/PEI (FBS/PEI/CplxPlt(1)), showed a decrease in staining due to cytotoxicity of un-complexed PEI.

### Example 2

### Expression of GFP Cochleates In Vivo

Green Fluorescent Protein (GFP) cochleates have been administered intravenously in order to examine their expression in vivo.

### Local Infection of Cochleates

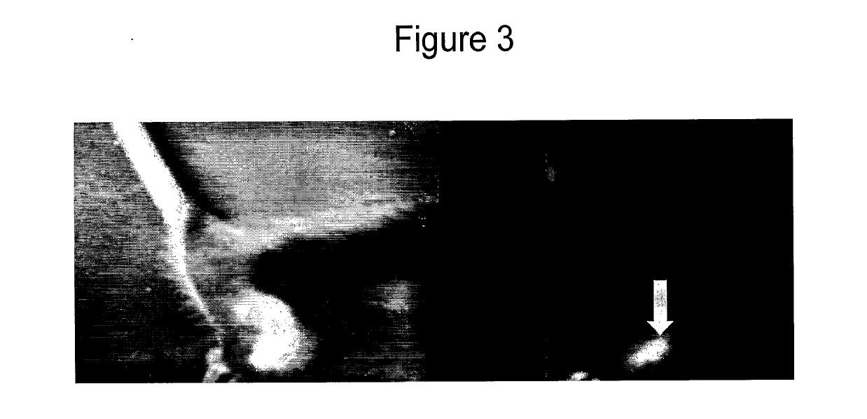

150 μg of GFP cochleates (500 nm-5 μm) were injected locally into the leg muscle of C57BL/6 mice. GFP expression was examined for the subsequent 7 days. After 3 days, the leg muscle shows GFP expression (see FIG. 3). GFP transgene expression is also seen for an additional four days.

### Intra-Tumour Injection of GFP Cochleates

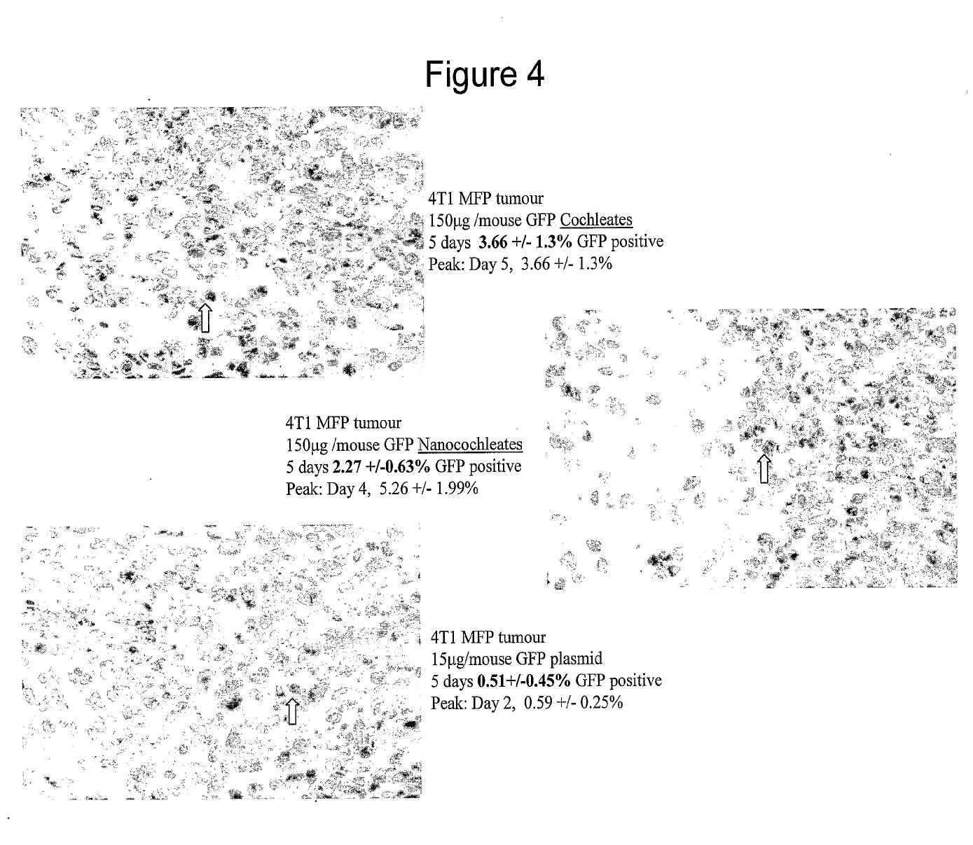

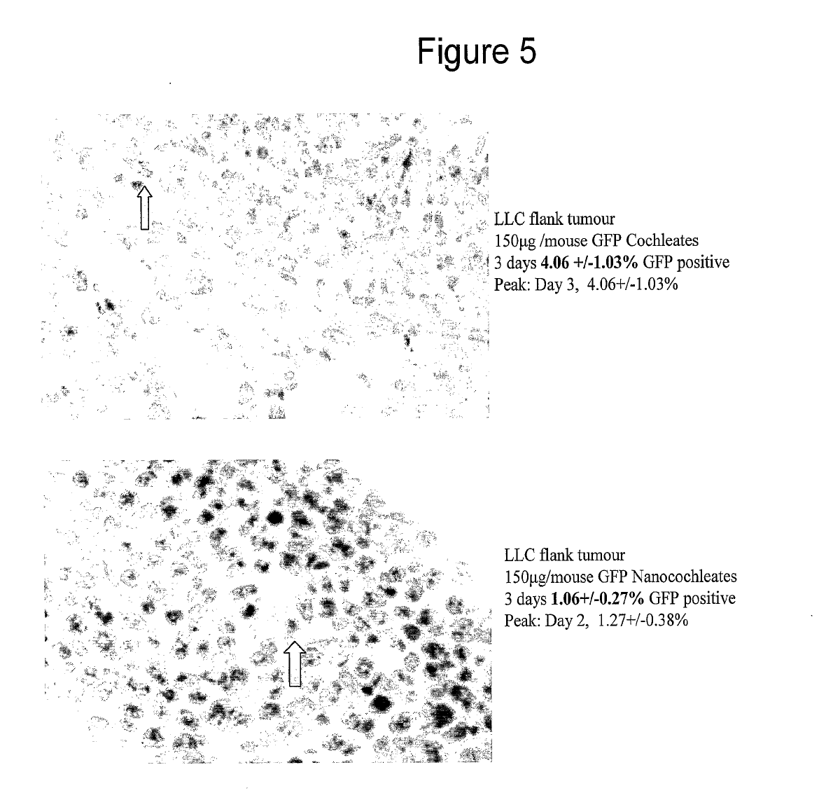

GFP cochleates (150 μg), GFP nanocochleates (300 nm-1 μm, 150 μg), empty cochleates, and unencochleated “naked” DNA (15 μg) were injected into two tumour models: Lewis Lung Carcinoma flank tumours and 4T1 adenocarcinoma mammary fat pad (MFP) tumours, and observed over a seven day period. Frozen sections of tumour and local lymph node were stained immunohistochemically for GFP expression. The GFP cochleates, GFP nanocochleates, and “naked” DNA all showed a positive stain for GFP expression in both tumour models, as shown in FIGS. 4 and 5.

### Intravenous Delivery of GFP Cochleates

DNA cochleates with and without casein, an aggregation inhibitor, were injected intravenously (150 μg PS/15 μg DNA) in order to deliver plasmid expressing GFP to mammary fat pad tumours. Gene transfer and expression were assayed using immunohistochemistry for GFP in frozen tumour sections 4 days subsequent to injection.

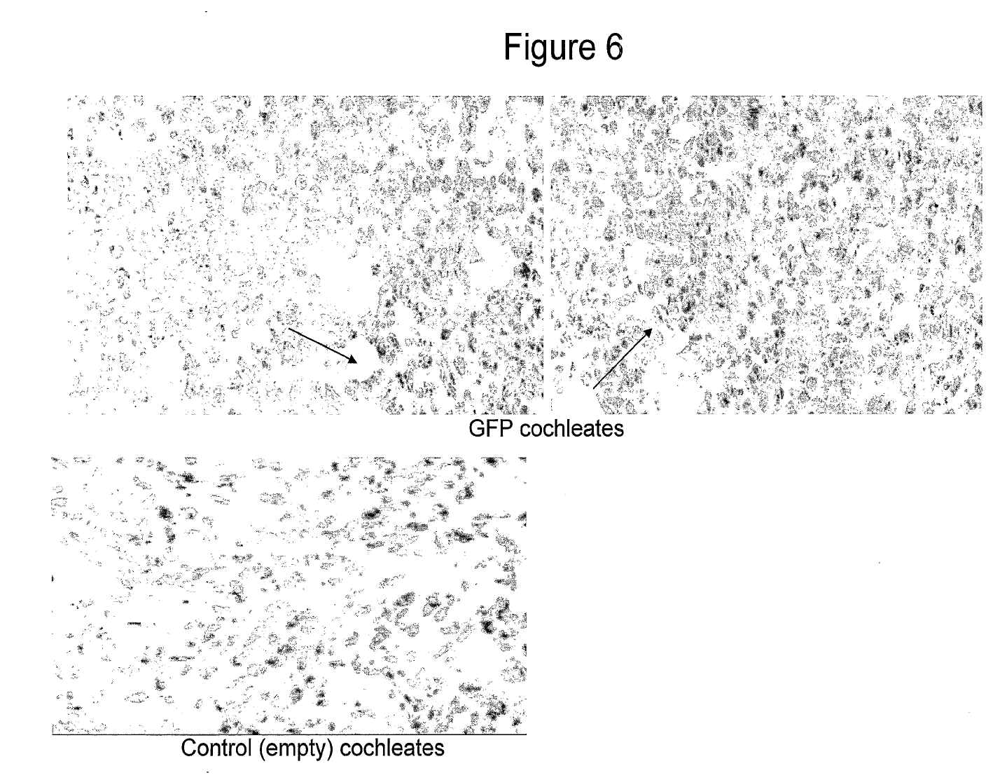

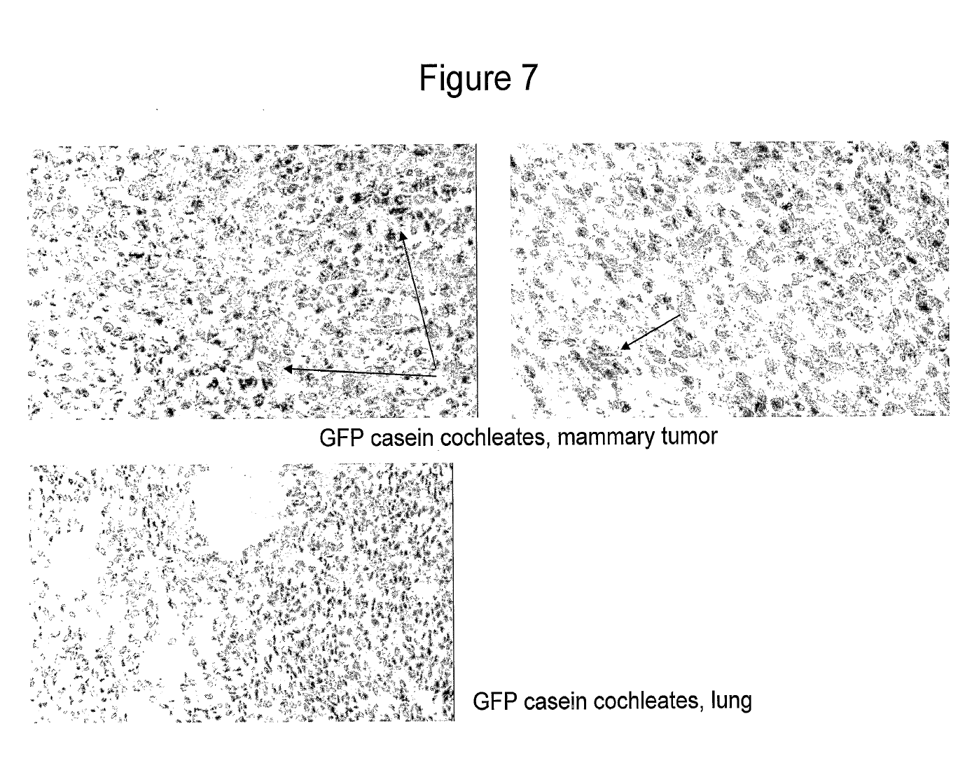

Preliminary data indicates that intravenous administration of GFP DNA cochleates mediates gene expression in mammary tumors, as shown in FIG. 6. Preliminary data indicates that intravenous delivery of casein nanocochleates results in significant amount of gene expression in the mammary tumor, but not the lungs, as shown in FIG. 7.

### Example 3

### Cytotoxicity of DNA Formulations In Vitro

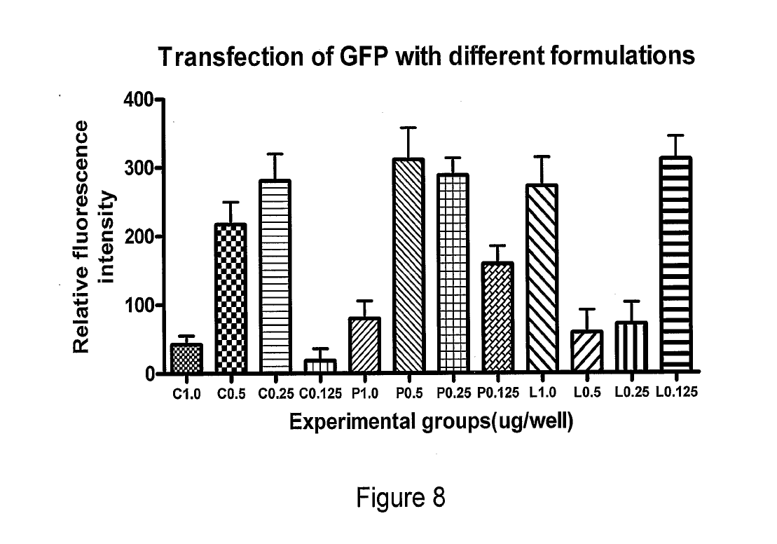

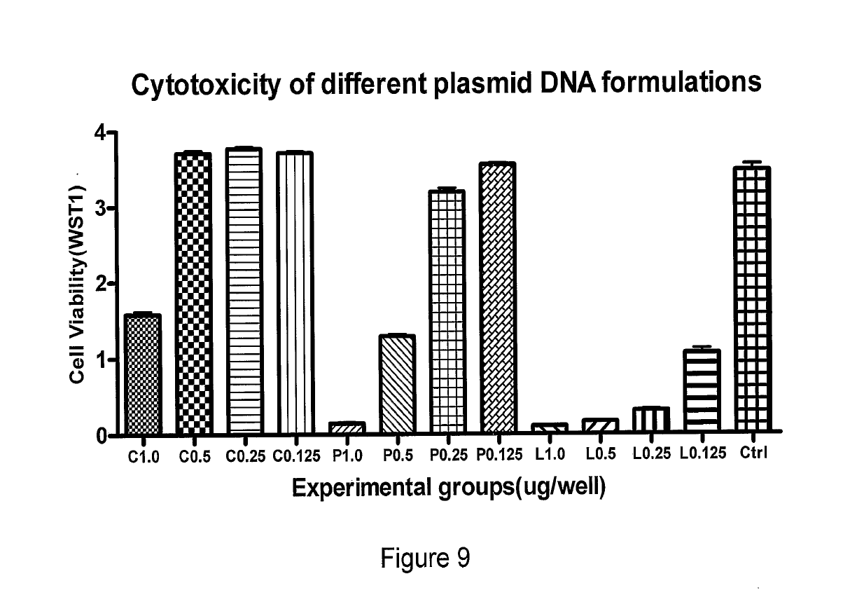

SKOV3 cells were transfected with DNA formulations (C: DNA cochleates formulated with polyethylenimine 25K linear (PEI). P: DNA PEI complex. L: DNA with Lipofectamine 2000. Ctrl: control, untreated cells) from 1.0 to 0.25 pg per well of DNA (FIG. 9). 72 hours after transfection, cell viabilities were tested (WST1 assay). The results (FIG. 9) demonstrate that cochleate/PEI DNA formulation have significantly lower cytotoxicity than those of PEI or Lipofectamine groups. Cells were lysed and tested for fluorescence intensities at 72 hours after transfection. In contrast to the lipofectamine and PEI/DNA formulations, the cochleate formulation gave equivalent transfection at the concentrations of 0.5 and 0.25 ug/well where there was no toxicity seen in WST1 assay (FIG. 8).

### Example 4

### Preparation of Lipid-Vesicles Containing Cross-Linked Peptide (Developed for the Preparation of Immunogenic Complexes)

### Initial Preparation of Peptide-Lipid Complex

Joining of crosslinking reagent to phosphatidylethanolamine was accomplished according to the method of Martin and Papahadjopoulos (J. Biol. Chem. 257:286-288 (1982)) as follows:

Succinimidyl-4-(p-maleimidophenyl)butyrate (SMPB), phosphatidylethanolamine (PE), anhydrous methanol, and triethylamine were reacted for two hours at room temperature under N₂ (e.g. 2 ml methanol, 5 μA triethylamine, 17 mg SMPB, added to 25 mg dry PE). Methanol was removed from the reaction products with a stream of N₂. 2 ml CHCl₃ was added under N₂ and the mixture was extracted 2 times with 2 ml of 1% NaCl in H₂O by centrifuging at 1500 RPM for 5 min, also under N₂. Chromatography on a silica gel column was then performed as follows:

The column was washed with 50 ml CHCl₃. The sample in 5 ml CHCl₃ was loaded on the column, followed by 10 ml each of CHCl₃:MeOH 40:1, 30:1, 25:1, 20:1, 15:1, and then 40 ml 10:1 while collecting 5 ml fractions. Thin layer chromatography of the silica gel column fractions (solvent—CHCl₃:MeOH:H₂O, 65:25:4) was then conducted. The coupled cross linker and phospholipid (MPB-PE) that eluted in the early 10:1, CHCl₃:MeOH fractions showed a single spot comigrating with a PE standard. The derivatized PE spot was ninhydrin negative, iodine positive. The PE standard spot was ninhydrin positive. Silica gel column fractions containing MPB-PE were pooled and stored under N₂ at −20° C. Phospholipid was quantitated by the method of Bartlett (J. Biol. Chem. 234:466 (1959)). The average yield was 50%.

### Reduction of Peptide Containing a Terminal Cysteine

The peptide prepared by solid-phase procedures (Yarney and Marrifield, in “The Peptide: Analysis, Synthesis, Biology.” Academic Press, N.Y. Vol. 2 1-284.) was dissolved in 1M acetic acid (HAc) (e.g. 2 mg peptide in 1 ml HAc). Dithiothreitol (DTT) was added to 100 mM conc. The sample was degassed, nitrogenized, and then incubated in a screw-cap tube at 37° for 3 hours. Affinity chromatography on Bondelute column (3 cc column obtained from Analytichem International C18, part 607203) was performed as follows:

The column was washed with 2 vols 100% MeOH, then 10 vols 1M HAc. The peptide was applied to the Bondelute column. The column was washed with 5 volumes 1M HAc, then 5 vols 0.02% trifluoracetic acid (TFA). The sample was eluted with 5 volumes 60% acetonitrile, 0.02% TFA. The eluted sample was lyophilized overnight and stored at −20° C. Essentially complete recovery was obtained.

Preparation of Immunogenic Composites Comprising Peptide-Lipid Complex Associated with a Mixture of Lipids and a Sterol (Cholesterol)

The lipids, MPB-PE, sphingomyelin (SP), phosphatidylcholine (PC), and phosphatidylserine (PS), and the sterol, cholesterol (Ch), were dissolved in ether (10 mg/ml) at a molar ratio of MPB-PE:SP:PC:PS:Ch of 2:1:1:1:5. The sample was dried under a nitrogen stream. The dried sample was resuspended in buffer (20 mM citric acid, 35 mM disodium phosphate, 108 mM NaCl, 1 mM EDTA, pH 4.5) at 4 mg lipid and cholesterol/ml. The sample was sonicated to form a particulate suspension. Octyl-β-D-glucoside (10 mg/mg lipid and sterol) was added to the sonicated sample. The sample was again sonicated to dissolve all lipid. The sample was stored under N₂ in the dark.

### Coupling of Peptide to Lipid Mixture Containing Derivatized Phosphatidyl-Ethanolamine (MPB-PE)

Premade reduced peptide from step II above was added to the lipid/sterol mixture from step III above containing MPB-PE (MPB-PE molar concentration was 2× molar concentration of peptide) at pH 4.5. The pH was adjusted to 6.5 with 1N NaOH. The MPB-PE and peptide in the mixture were allowed to react under N₂ at room temperature overnight. The mixture was dialyzed against several changes of phosphate buffered saline at 4° C., pH 7.2. The immunogenic composite comprising the protein-lipid complex associated with a mixture of lipids and cholesterol was recovered from the dialysis bag and stored refrigerated (4° C.).

### EQUIVALENTS

Those skilled in the art will recognize, or be able to ascertain using no more than routine experimentation, many equivalents to the specific embodiments of the invention described herein. Such equivalents are intended to be encompassed by the following claims.

1. (canceled)

2. A nucleotide-cochleate composition comprising:
a cochleate;
a lipophilic tail; and
a siRNA associated with the cochleate;
wherein the siRNA is complexed with a transfection agent and covalently bound to N-hydroxysuccinimidyl 3-(2-pyridyldithio)propionate (SPDP), and
wherein the SPDP is covalently bound to the lipophilic tail.

3. (canceled)

4. The composition of claim 2, wherein the SPDP stabilizes the siRNA.

5. The composition of claim 2, wherein the SPDP facilitates association of the siRNA with the cochleate component.

6. The composition of claim 2, wherein the cochleate comprises a negatively charged lipid component and a multivalent cation component.

7. The composition of claim 2, wherein the cochleate comprises soy phosphatidylserine.

8. (canceled)

9. The composition of claim 2, wherein the linker is N-succinimidyl-4-(p-maleimidophenyl)butyrate (SMPB).

10-11. (canceled)

12. The composition of claim 2, wherein the nucleotide is a morpholino oligonucleotide.

13. The composition of claim 12, wherein the morpholino oligonucleotide is an antisense morpholino oligonucleotide.

14. The composition of claim 2, wherein the nucleotide is a short double-stranded DNA.

15. The composition of claim 2, wherein the nucleotide is a ribozyme.

16. The composition of claim 2, wherein the nucleotide is an aptamer.

17. The composition of claim 2, wherein the nucleotide is a transcription factor decoy.

18. The composition of claim 2, wherein the siRNA comprises at least one mismatch.

19. The composition of claim 2, wherein the siRNA comprises at least one substitution.

20. The composition of claim 2, wherein the siRNA is between about 18 and about 25 nucleotides long.

21. The composition of claim 2, wherein the siRNA is between about 21 and about 23 nucleotides long.

22. The composition of claim 2, wherein the siRNA mediates RNA interference against a target mRNA.

23. The composition of claim 22, wherein the target mRNA expresses a protein selected from the group consisting of: a cancer protein, a virus protein, an HIV protein, a fungus protein, a bacterial protein, an abnormal cellular protein, and a normal cellular protein.

24. The composition of claim 2, wherein the siRNA mediates inhibition of translation of a target mRNA.

25. The composition of claim 24, wherein the target mRNA expresses a protein selected from the group consisting of: a cancer protein, a virus protein, an HIV protein, a fungus protein, a bacterial protein, an abnormal cellular protein, and a normal cellular protein.

26. The composition of claim 2, further comprising a second nucleotide directed against a second target mRNA.

27. The composition of claim 2, wherein the siRNA is complexed with a transfection agent prior to contacting a liposomes.

28. The composition of claim 27, wherein the transfection agent is a polycationic transfection agent.

29. The composition of claim 27, wherein the transfection agent is polyethylenimine (PEI), protamine, or a derivative thereof.

30. (canceled)

31. A method of administering a nucleotide to a host comprising:
administering a biologically effective amount of a nucleotide-cochleate composition according to claim 2 to a host.

32. A method of treating a subject having a disease or disorder associated with expression of a target mRNA, comprising: administering to a subject a therapeutically effective amount of an nucleotide-cochleate composition, comprising a cochleate and a nucleotide directed against a target mRNA associated with a disease or disorder, wherein the nucleotide is bound to a lipophilic tail via a linker, such that the disease or disorder is treated.

33-37. (canceled)

38. The composition of claim 33, wherein the nucleotide is a morpholino oligonucleotide.

39. The composition of claim 38, wherein the morpholino oligonucleotide is an antisense morpholino oligonucleotide.

40. The composition of claim 33, wherein the nucleotide is a short double-stranded DNA.

41. The composition of claim 33, wherein the nucleotide is a ribozyme.

42. The composition of claim 33, wherein the nucleotide is an aptamer.

43. The composition of claim 33, wherein the nucleotide is a transcription factor decoy.

44-53. (canceled)

54. A method of administering a nucleotide to a host comprising:
administering a biologically effective amount of a nucleotide-cochleate composition according to claim 33 to a host.

55. A method of treating a subject having a disease or disorder associated with expression of a target mRNA, comprising: administering to a subject a therapeutically effective amount of a nucleotide-cochleate composition, comprising a cochleate and a nucleotide directed against a target mRNA associated with a disease or disorder, wherein the nucleotide is complexed to a transfection-agent, such that the disease or disorder is treated.
